(19) United States
(12) Patent Application Publication
(10) Pub. No.: US 2023/0380967 A1
(43) Pub. Date: Nov. 30, 2023

(54) Method and Apparatus for Loading and Delivering a Prosthetic Heart Valve

(71) Applicant: Cephea Valve Technologies, Inc., Abbott Park, IL (US)

(72) Inventors: Russell Shelton, San Jose, CA (US); Randolf Von Oepen, Aptos, CA (US); Gregory M. Hyde, Menlo Park, CA (US)

(73) Assignee: Cephea Valve Technologies, Inc., Abbott Park, IL (US)

(21) Appl. No.: 18/322,168

(22) Filed: May 23, 2023

Related U.S. Application Data
(60) Provisional application No. 63/347,096, filed on May 31, 2022.

(51) Int. Cl. A61F 2/24

(52) U.S. Cl. CPC A61F 2/2433

(57) ABSTRACT

A system for heart valve repair includes a collapsible and expandable replacement heart valve and a delivery device for implanting the implanting the valve. The delivery device includes a base unit positioned at a proximal end of the delivery device, an outer catheter extending from the base unit, a first inner catheter positioned within the outer catheter, a second inner catheter positioned within the first inner catheter, a mandrel positioned within the second inner catheter, and a nosecone adapted to be couple to the second inner catheter.

### CROSS-REFERENCE TO RELATED APPLICATIONS

This application claims priority to U.S. Patent Application No. 63/347,096, filed May 31, 2022, the disclosure of which is hereby incorporated by reference herein.

### BACKGROUND OF THE DISCLOSURE

A primary treatment for heart valve disease is valve replacement. One form of replacement device is a bio-prosthetic replacement valve, particularly those designed for a minimally invasive procedure. To properly deliver a replacement valve in a minimally invasive procedure, the valve must endure a number of steps which all must be executed accurately and effectively to successfully deliver and implant the replacement valve in the region of the native valve. For example, the replacement valve may need to be packaged, unpackaged, collapsed, loaded into a delivery device, delivered to the native valve, deployed and implanted, all while avoiding error or unexpected results in each step of the process.

Certain unintended results might occur throughout the collapsing and loading process. A collapsible and expandable replacement valve might not easily collapse in a uniform manner, which may result in an unbalanced or unexpected deployment from the delivery device inside the heart. Further, the replacement heart valve may have a particular shape or components on the valve that increase the difficulty of the collapsing and loading processes.

Thus, further developments in the field of collapsing and loading a replacement heart valve into a delivery device are desired.

### BRIEF SUMMARY OF THE DISCLOSURE

According to a first aspect of the disclosure, a system for loading a replacement heart valve into a delivery device includes a base unit, a first catheter, a mandrel, a hypotube, and a counterbearing. The base unit may be positioned at a proximal end of the delivery device. The first catheter may be adapted to be coupled to and extend distally from the base unit, and the first catheter may define a first lumen. The mandrel may be adapted to be coupled to and extend distally from the base unit, and the mandrel may be sized to be positioned radially inward of the first catheter and extend through the first lumen. The hypotube may be configured to be coupled to a distal end of the first catheter, wherein the hypotube includes an internal support member coupled thereto configured to support a replacement heart valve during a collapsing of the replacement heart valve. The counterbearing may be adapted to be coupled to a distal end of the mandrel to maintain axial positioning of the internal support member relative to the replacement heat valve while the internal support member supports a collapse of the replacement heart valve.

According to a second aspect of the disclosure, an internal support member for assisting with collapsing a replacement heart valve may include a first tube, a second tube, and a balloon. The first tube may extend from a proximal end to a distal end. A second tube may be coupled to the first tube at the distal end. The balloon may extend from a proximal end to a distal end, wherein the proximal end of the balloon may be coupled to the first tube and the distal end of the balloon may be coupled to a second tube. The balloon may be configured to be inflated and deflated via injection and withdrawal of a fluid. The proximal end of the first tube may be adapted to be coupled to a catheter.

According to a third aspect of the disclosure, a method of loading a replacement heart valve into a delivery device may include passing a mandrel through a lumen of a replacement heart valve; coupling a first inner catheter to a retention mechanism coupled to the replacement heart valve; positioning a second inner catheter over the mandrel such that the mandrel passes through a lumen of the second inner catheter, where the second inner catheter extends within the first inner catheter; positioning a hypotube over the mandrel such that the mandrel passes through a lumen of the hypotube; coupling the hypotube to the second inner catheter; translating the first inner catheter in a proximal direction to draw the replacement heart valve through a first funnel to transition the replacement heart valve from an expanded condition to a collapsed condition over an internal support member coupled to the hypotube; translating the first inner catheter in the proximal direction to draw the replacement heart valve from the first funnel to a second funnel; decoupling the first funnel from the second funnel; translating the first inner catheter in the proximal direction to draw the replacement heart valve from the second funnel to an outer catheter; decoupling the second funnel from the outer catheter; coupling a nosecone to the second inner catheter; and translating the second inner catheter proximally relative to the outer catheter to seat the nosecone within an opening of the outer catheter.

According to a fourth aspect of the disclosure, a method of collapsing a replacement heart valve may include passing a mandrel through a lumen of a funnel; coupling an inner catheter to the replacement heart valve; positioning a hypotube over the mandrel such that the mandrel passes through a lumen of the hypotube, the hypotube including an internal support member coupled thereto; drawing the inner catheter proximally to translate the replacement heart valve through the funnel to transition the replacement heart valve from an expanded condition to a collapsed condition while the internal support member is positioned radially inward of the replacement heart valve.

### BRIEF DESCRIPTION OF THE DRAWINGS

FIG. 1A is a front view of a packaging assembly according to an embodiment of the disclosure.

FIG. 1B is a cut-away view of the packaging assembly of FIG. 1A.

FIG. 1C is an exploded view of the packaging assembly of FIG. 1B in which the replacement heart valve is covered by a fabric skirt.

FIG. 2A is a perspective view of a frame of a replacement heart valve that may be used with the packaging assembly of FIG. 1A.

FIG. 2B is an enlarged view of an attachment member of the replacement heart valve of FIG. 2A.

FIGS. 3A-3C are perspective, top and front views, respectively, of the ring of the packaging assembly of FIG. 1A.

FIG. 3D is a perspective view of a first ring portion of the ring of FIG. 3A.

FIGS. 4A-4C are perspective, bottom, and front views, respectively, of the valve support of the packaging assembly of FIG. 1A.

FIG. 4D is an enlarged view of a portion of the valve support of FIG. 4A.

FIG. 5 is a perspective view of a funnel of the packaging assembly of FIG. 1A.

FIGS. 6A-6B are perspective and front views, respectively of the packaging assembly of FIG. 1A without a funnel.

FIG. 6C is a perspective view of the packaging assembly of FIG. 6A without a cap, collar, and sutures of a retention mechanism and without a frame of the prosthetic heart valve.

FIG. 7A is a perspective view of the replacement heart valve of FIG. 2A with a first ring portion positioned around the valve.

FIG. 7B is a perspective view of the replacement heart valve of FIG. 2A with a ring positioned around the valve.

FIG. 8A-8B are front and top views, respectively, of the packaging assembly of FIG. 1A without a valve support.

FIG. 9 is a front transparent view of the packaging assembly of FIG. 1A without a ring.

FIG. 10 schematic view of a prosthetic heart valve delivery device according to an embodiment of the disclosure.

FIGS. 11A-11N show steps of removing the packaging assembly of FIG. 9 from packaging and transitioning the replacement heart valve from an expanded condition to a collapsed condition through the funnel of FIG. 5.

FIG. 11N-1 is a side schematic view of a hypotube with a balloon coupled thereto to uniformly collapse the replacement heart valve through the slotted funnel.

FIG. 11N-1′ is a side schematic view of a hypotube with a balloon coupled thereto according to another embodiment of the disclosure

FIG. 11N-1″ is a side schematic view of a hypotube with a balloon coupled thereto according to another embodiment of the disclosure.

FIGS. 11N-2 and 11N-3 are superior and side schematic views, respectively, of a mandrel and counterbearing used in a process of loading the replacement heart valve into the delivery device.

FIGS. 11O-11S show steps of transitioning the replacement heart valve from an expanded condition to a collapsed condition through the funnel of FIG. 5 continued from FIG. 11N.

FIGS. 12A-12K show steps of loading the replacement heart valve into the delivery device of FIG. 10.

FIG. 12L is a schematic side view of a replacement heart valve disposed within a delivery device according to another embodiment of the disclosure.

FIG. 12M is a schematic side view of a nosecone of the delivery device of FIG. 12L.

FIGS. 12N-12O are schematic side views of the nosecone of FIG. 12M with a balloon in deflated and inflated configurations, respectively.

FIGS. 13A-13F show steps of deploying the replacement heart valve from the delivery device of FIG. 10.

FIGS. 14A-14B are perspective views of a window funnel according to an embodiment of the disclosure.

### DETAILED DESCRIPTION

The packaging assemblies of the present disclosure hold a replacement heart valve securely in a shipment jar or packaging container and optionally allow for pre-tensioning of components of the replacement valve assembly such that the replacement valve is secured and tensioned in the packaging during shipment. The packaging assembly further allows the valve to be transitioned from a packaged (e.g., partially or fully expanded) configuration to a delivery (e.g., collapsed) configuration by translating the valve through a slotted funnel included in the packaging assembly to be inserted into a delivery device and prepared for delivery. The slotted funnel may be constructed as part of the packaging assembly, which serves to protect the replacement valve within the packaging and also reduces the complexity of the procedure when loading the valve into a delivery device.

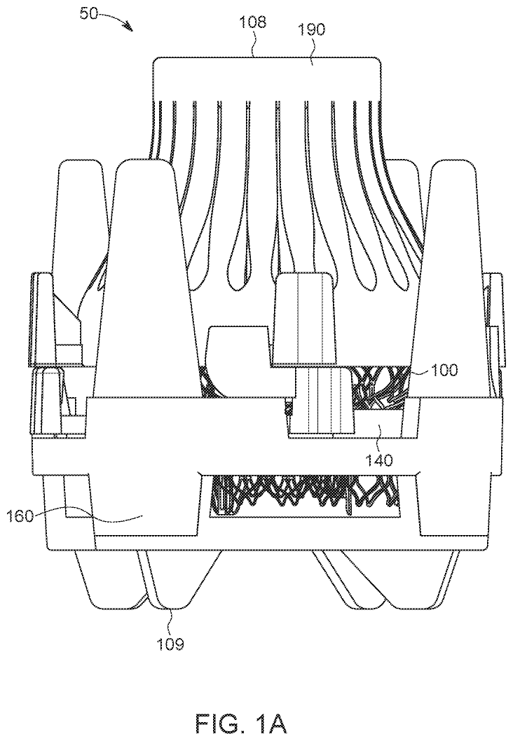

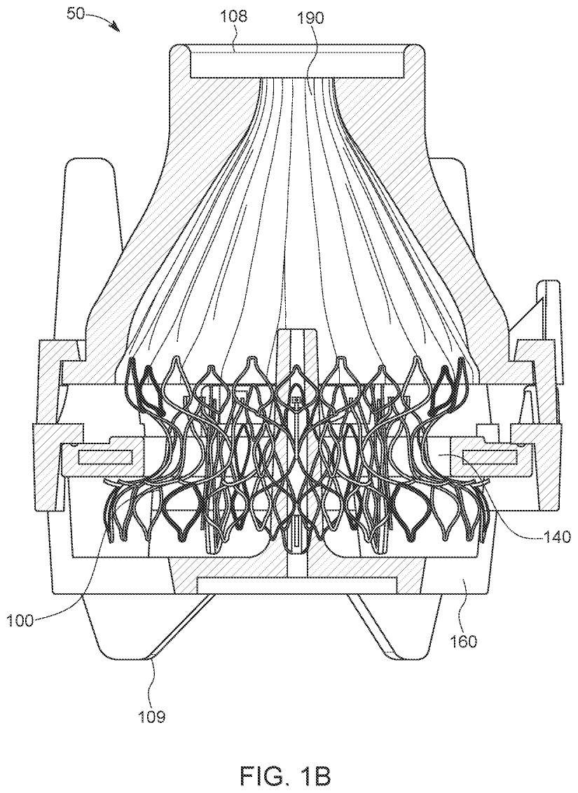

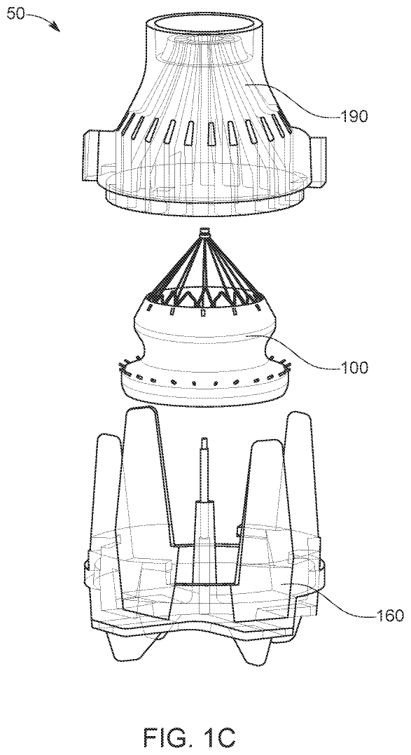

A fully assembled packaging assembly 50 according to an embodiment of the disclosure is shown in FIGS. 1A-1B. Packaging assembly 50 includes replacement heart valve 100, valve support 160, ring 140, and a first funnel or slotted funnel 190. Each component is described below in detail. For ease of illustration throughout the disclosure, packaging assembly 50 is described herein extending from proximal end 108 to distal end 109, wherein when the valve is being transitioned from the packaging assembly to a delivery device, the assembly will be oriented such that the proximal end abuts the delivery device and the distal end is farther away from the opening of the delivery device. In the particular illustrated example, the proximal end is synonymous with an inflow end of the replacement heart valve 100, and the distal end is synonymous with an outflow end of the replacement heart valve 100. However, it should be understood that the terms proximal and distal are solely used for convenience, and need not correspond to any particular directionality unless otherwise noted herein. For a clearer view of the components of packaging assembly 50, FIG. 1C shows an exploded view of the assembly, although it should be noted that the exploded view does not include ring 140. Certain embodiments described in the present disclosure may include a ring, and some may not.

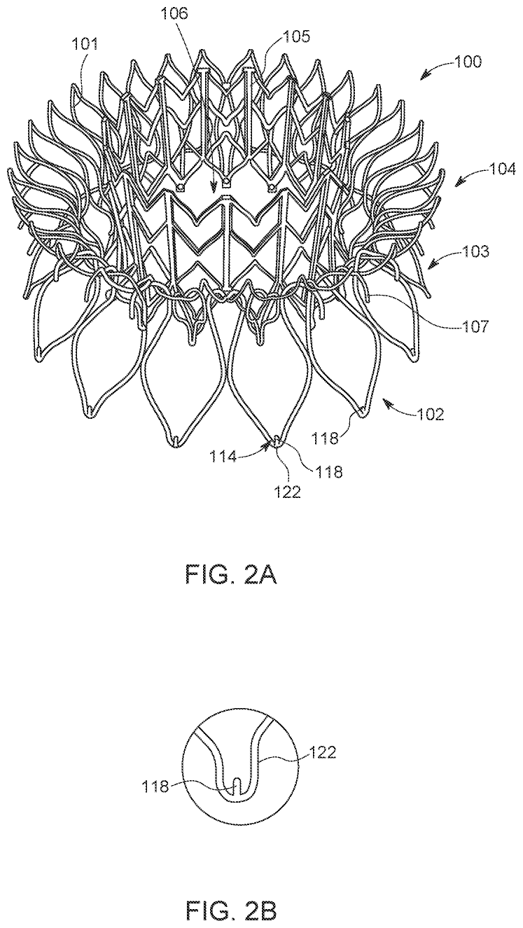

The packaging assemblies of the present disclosure may be used with replacement heart valves, such as replacement heart valve 100 shown more clearly in FIGS. 2A-2B, which is an expandable (e.g. self-expandable) prosthetic implant having an expanded configuration and a collapsed configuration. Replacement heart valve 100 may be a replacement mitral valve having a stent that includes an anchor assembly (e.g. for anchoring the replacement heart valve 100 within the native valve annulus) and a strut frame (e.g. for supporting prosthetic valve leaflets) disposed within the anchor assembly. However, replacement heart valve 100 may be suitable for use in replacing other native heart valves, such as the tricuspid valve, aortic valve, or pulmonary valve, although it may be best suited for replacing the atrioventricular valves. Anchor assembly 101 includes atrial anchor 102, ventricular anchor 104 and central portion 103 positioned axially between the atrial and ventricular anchors. Atrial anchor 102 is configured to be positioned on an atrial side of a mitral valve annulus, and ventricular anchor 104 is configured to be positioned on a ventricular side of the mitral valve annulus. Within packaging assembly 50, atrial anchor 102 is positioned relatively closer to proximal end 108 and ventricular anchor 104 is positioned relatively closer to distal end 109. Anchor assembly 101 may have an hour glass shape in that each of atrial anchor 102 and ventricular anchor 104 flares radially outward of central portion 103, such that the central portion defines a waist between the atrial anchor and the ventricular anchor. Anchor assembly 101 includes tines 107 having traumatic tips and extending generally radially outward proximate to ventricular anchors 104. Tines 107 promote secure implantation of valve 100 into a native valve annulus of a patient by anchoring into the surrounding tissue. Replacement heart valve 100 may also include strut frame 105 positioned radially inward of anchor assembly 101 and formed of a plurality of connected struts. The radially inner surfaces of strut frame 105 define a perimeter of central opening 106, which allows blood to flow through replacement heart valve 100 in the antegrade direction. Exemplary replacement heart valves are described in U.S. Pat. No. 10,470,881, the disclosure of which is hereby incorporated by reference herein in its entirety.

Replacement heart valve 100 includes one or more prosthetic leaflets (not shown). The leaflets may be secured to an interior of strut frame 105 and may be disposed at least partially in central opening 106. The prosthetic leaflets are configured to coapt with each other in order to control blood flow therethrough, allowing blood to flow from in a direction from atrial anchor 102 toward ventricular anchor 104 (the antegrade direction), but to substantially block from flowing in the opposite (retrograde) direction. The inner and/or outer surfaces of each of anchor assembly 101 and strut frame 105 may be partially or fully covered by cuffs or skirts, including those of fabric and/or tissue materials. An exemplary outer fabric skirt is shown on valve 100 in FIG. 1C.

Replacement heart valve 100 includes a delivery device attachment mechanism. For example, at least one atrial tip 114 of atrial anchor 102 forms crest 122 having attachment members 118, such as tines or pins, to which suture loops can be secured, shown in FIGS. 2A and 2B. In some embodiments, replacement heart valve 100 includes a plurality of attachment members 118. These attachment members 118 can be straight hooks, curved hooks, pins, tines, or other structures extending from a strut or member of the valve and to which a suture loop can be securely connected during shipment and subsequent delivery of the valve during implantation of the replacement heart valve 100. Attachment members 118 are sized and shaped to allow release of the suture loops from the attachment members and thus the valve after deployment of the valve within the patient. Thus, in the illustrated embodiment of FIG. 2B, attachment member 118 is in the form of a pin provided at the apex of each crest 122, with a free end extending toward the ventricular or outflow end of the replacement heart valve 100. Other exemplary structures that may be suitable for use as attachment member 118 are described in greater detail in U.S. Provisional Patent Application No. 63/171,148, filed Apr. 6, 2021 and titled “Frame Features for Transcatheter Mitral Valve Replacement Device,” the disclosure of which is hereby incorporated by reference herein.

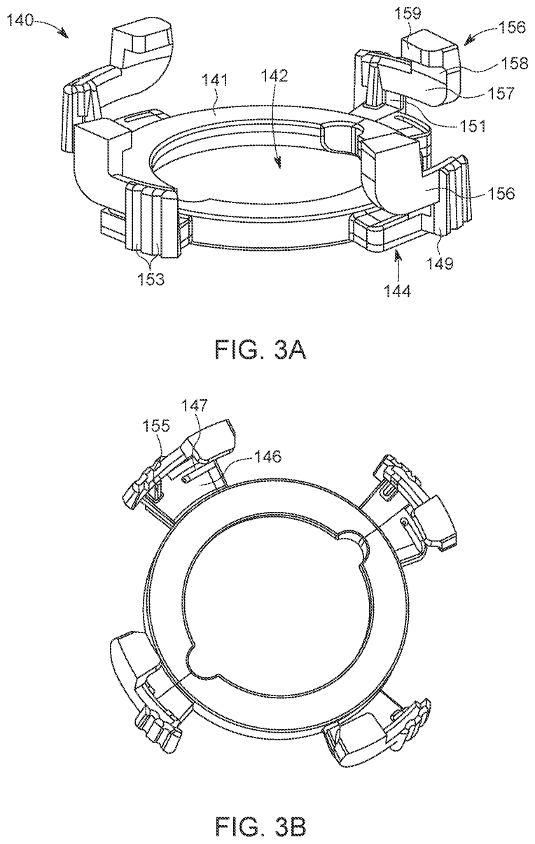

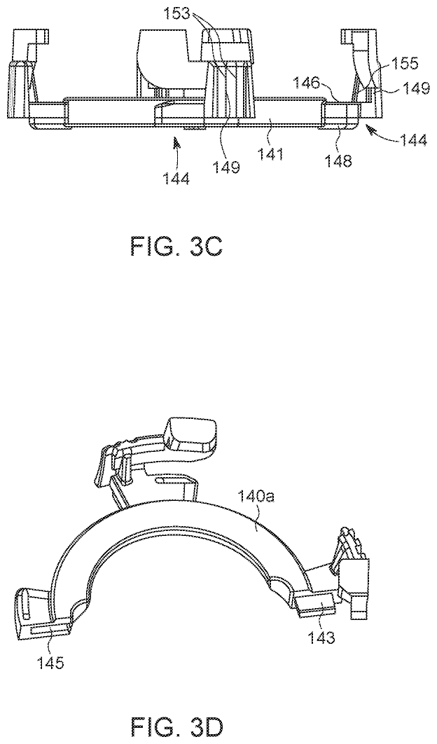

Ring 140 is described in further detail with reference to FIGS. 3A-3D. Ring 140 includes body 141 having an annular or circular shape and defining opening 142 therethrough. Ring 140 removably attaches to valve support 160 with an attachment mechanism. In the illustrated embodiment, the attachment mechanism is in the form of four spaced apart locking tabs 144 extending radially outward from body 141 and spaced around the circumference of the ring. As in the illustrated embodiment, locking tabs 144 may be evenly spaced apart around the circumference of body 141. Locking tabs 144 may be spaced about 90 degrees from adjacent locking tabs, as shown in FIG. 3B. In other examples, the locking tabs may be arranged at different intervals around body 141 relative to one another. In FIGS. 3A and 3B, ring 140 includes four locking tabs 144, although in other examples there may be more or fewer locking tabs.

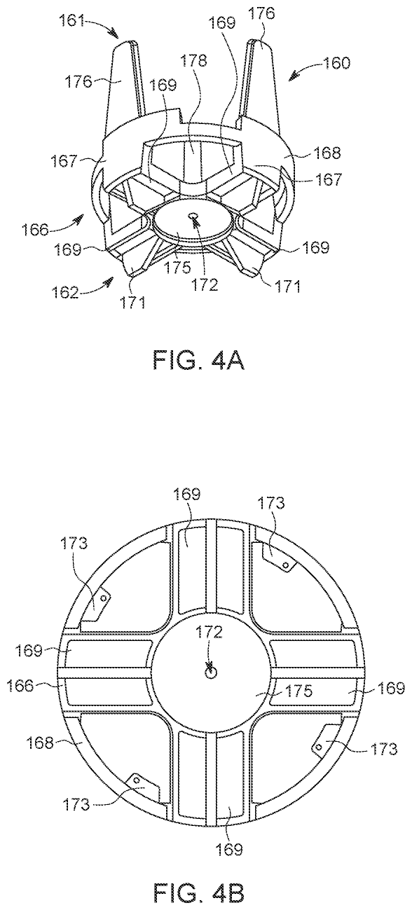

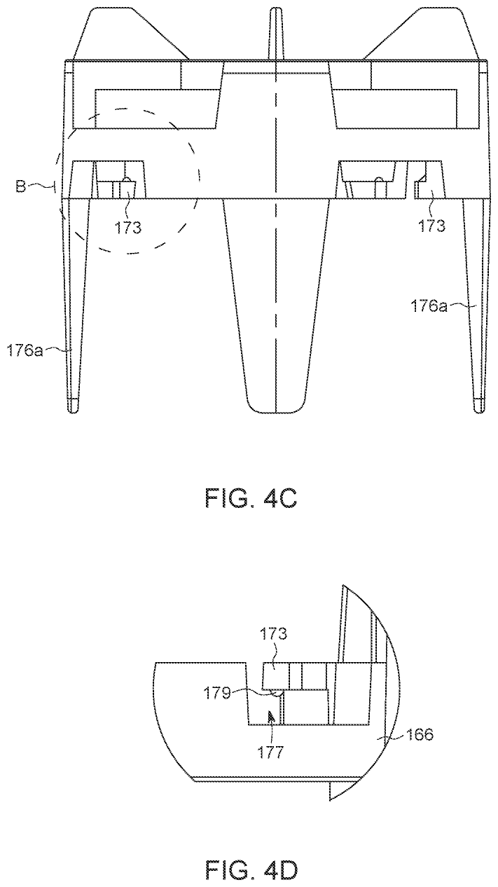

As shown in FIG. 3C, each locking tab 144 includes a proximal surface 146 and opposing distal surface 148. Proximal and distal surfaces 146, 148 may be substantially planar. Locking tab 144 includes primary extension 149 that extends the furthest radially outward of the locking tab 144. Primary extension 149 has a generally rectangular shape with the length extending in the proximal-distal direction, as best shown in FIG. 3C. Although, in other examples the primary extensions may be trapezoidal, round, triangular and are not limited to the shape shown in the figures. Primary extension 149 may also include ridges 153 on an outer surface thereof. In the illustrated embodiment, ridges 153 extend in the proximal-distal direction and may facilitate a user in readily gripping primary extensions 149 to manipulate (e.g. rotate) ring 140. It should be understood that additional or alternative texturizations may be provided to assist with manipulation and/or gripping of portions of ring 140. A ramp 155 may be positioned between primary extension 149 and body 141, and in the illustrated embodiment is connected to inner surface 151 (FIG. 3A) of primary extension 149 and proximal surface 146 to provide structural support for extension 149. Ramp 155 has a substantially triangular shape as shown in FIGS. 3A and 3C, which provides support to primary extension 149. Proximal surface 146 also includes a groove 147 shown in FIG. 3B for receiving a corresponding projection 179 (FIG. 4D) of the valve support to form a snap fit connection with the valve support, described in greater detail below.

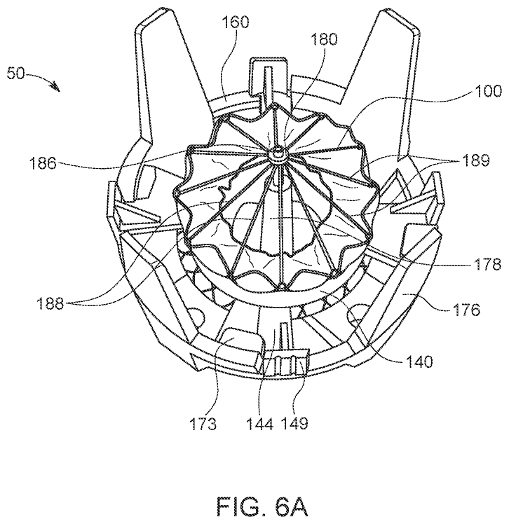

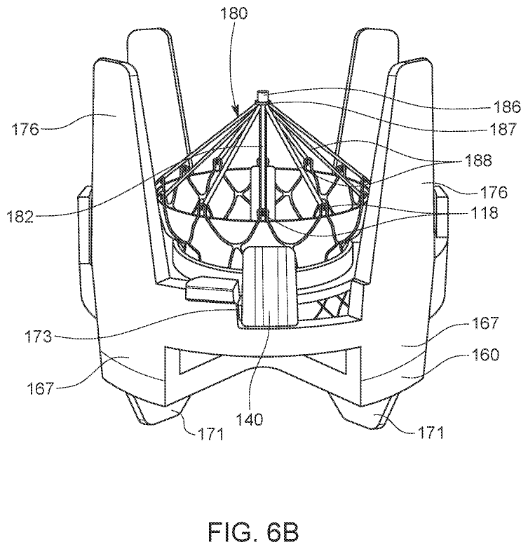

Locking tabs 144 may allow ring 140 to be moved via rotation from an unlocked position to a locked position in which ring 140 is rotationally and axially locked with valve support 160. Such connection is described in further detail below in connection with additional descriptions of FIGS. 6A-6C. Primary extensions 149 may provide a hard stop feature to prevent over-rotation of ring 140 within valve support 160. Coupled to primary extensions 149 are secondary extensions 156, which include elongate portion 157 extending in a direction generally parallel to the circumference of body 141, perpendicular portion 158 extending in a proximal direction perpendicular to the elongate portion such that the elongate and perpendicular portions generally form an L-shape, and overhang 159 extending inwardly from the perpendicular portion. Secondary extensions 156 allow for the secure coupling of ring 140 to funnel 190, as is described below in greater detail.

Ring 140 may be modular, made up of two half pieces, first ring portion 140a and second ring portion 140b, which may be adapted to mate with each other to form ring 140. Each ring portion 140a, 140b includes male protrusion 143 extending from body 141 and locking tab 144 on a first end of the ring portion and a corresponding female aperture 145 defined by the body and locking tab on the opposing end of the ring portion, as shown on first ring portion 140a in FIG. 3D. Protrusion 143 and aperture 145 of first ring portion 140a are positioned opposite those of second ring portion 140b, such that the protrusion of the first ring portion mates with the aperture of the second ring portion, and the protrusion of the second ring portion mates with the aperture of the first ring portion to securely, but detachably, couple the ring portions to one another to form ring 140. It is contemplated that ring 140 may be formed into any number of modular pieces (or otherwise a single non-modular piece) that may be attached to each other in the manner described above to form a full ring, such as 3 portions, 4 portions, etc.

Valve support 160 is described in further detail with reference to FIGS. 4A-4D. Valve support 160 has proximal end 161 and distal end 162 and includes base 166 near the distal end. Base 166 includes outer rim 168 with four platforms 169 extending distally from rim 168 and connected to the rim by a respective arm 167, platforms which extend across rim 168 to form a “x’- or “cross”-shape such that outer rim 168 is radially outward of platforms 169, as shown in FIGS. 4A and 4B. A proximal surface of base 166 may include ridges along the surface to provide rigidity to the base. Platforms 169 connect to each other at central portion 175. The central portion 175 may be generally circular and may define a central opening 172 for receiving a pin 182 (shown in FIG. 6C) and/or a guidewire or other alignment tool therethrough. Each platform 169 may have a respective rib 171 extending distally from a distal surface of base 166, and ribs 171 may be positioned on respective platforms 169 between the outer diameter of the base and central portion 175. Ribs 171 may provide locations for the user to grasp valve support 160 to lift the valve support out of the jar, for example without sacrificing sterility of the replacement heart valve or contacting more sensitive components.

Base 166 includes a plurality of fins 176 extending proximally from outer rim 168 which align with a respective one of the plurality of arms 167. Each fin 176 includes lateral side walls which taper inward in the proximal-distal direction such that the fin has a generally trapezoidal shape. Fins 176 are designed to provide rigidity to valve support 160 and protect replacement heart valve 100 from the walls of the jar or container during shipment. In this regard, fins 176 may be any shape including round, rectangular, triangular, which would enable the fins to provide rigidity to the structure. In this embodiment, there are four fins 176 equally spaced around the circumference of outer rim 168 of valve support 160 with the fins being spaced about 90 degrees apart from adjacent fins. Accordingly, the four arms 167 are also spaced apart from adjacent arms about 90 degrees. As shown in FIG. 4C, adjacent a side wall 176a of each fin 176 is ledge 173 projecting radially inward of outer rim 168 to define groove 177 between the ledge and outer rim for receiving a portion of locking tab 144. Further, ledge 173 includes projection 179 sized and shaped to engage groove 146 of locking tab 144 to form a snap fit connection. This connection rotationally and axially locks ring 140 to valve support 160. Ledge 173 and groove 177 are best shown in FIGS. 4C and 4D. When locking tab 144 of ring 140 is positioned within groove 177 of valve support 160, extension 149 may abut a side surface of ledge 173 to prevent further rotation of ring 140, as shown in FIG. 6B.

With reference to FIG. 4A, valve support 160 further includes cannulated rod 178 defining a central lumen (not shown) which aligns with central opening 172 of central portion 175 of base 166, the lumen configured to receive pin 182 of retention mechanism 180. When replacement heart valve 100 is packaged within packaging assembly 50, cannulated rod 178 is positioned extending through central opening 106 of the replacement heart valve, and also through an opening between the prosthetic leaflets disposed within support frame 105. Rod 178 has an exterior surface that is advantageously smooth to prevent abrasion or damage to the replacement heart valve, including abrasion to the prosthetic leaflets.

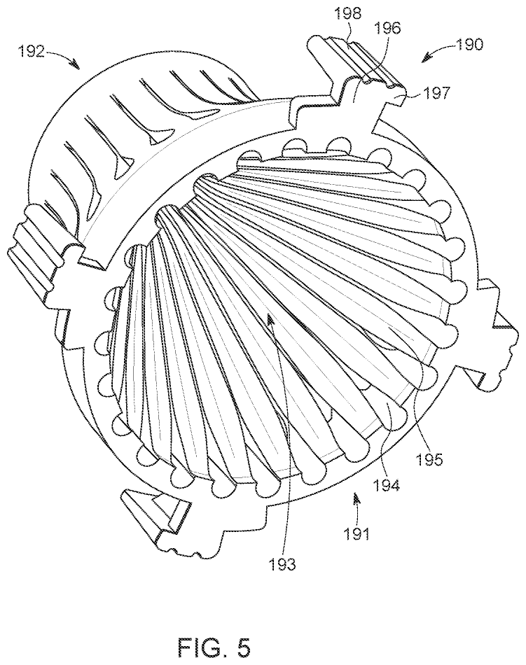

Slotted funnel 190 is described in further detail with reference to FIG. 5. Slotted funnel 190 defines a length extending from entry end 191 to exit end 192, wherein the entry end is nearer distal end 109 and the exit end is nearer proximal end 108 of packaging assembly 50 when in the fully assembled configuration. Slotted funnel 190 defines lumen 193 therethrough, the lumen having a tapering diameter corresponding to an inner surface of the funnel. That is, lumen 193 may have the same diameter as the inner surface of slotted funnel 190 along any plane extending perpendicular to a longitudinal axis along which the funnel extends. When slotted funnel 190 is used as intended, a replacement valve, such as valve 100, may be translated through the funnel in a loading direction, entering the funnel at entry end 191 and emerging from the funnel at exit end 192. Slotted funnel 190 has a first diameter at entry end 191 and a second diameter at exit end 192, the second diameter being smaller than the first diameter. The diameter of slotted funnel 190 may taper as the funnel extends from entry end 191 to exit end 192. In some examples, the rate at which the diameter of slotted funnel 190 tapers may vary such that the diameter tapers more rapidly near entry end 191 than it tapers near exit end 192. In other examples, the rate at which the diameter tapers may be consistent for the full length of the funnel. The second diameter at exit end 192 may be equal to or smaller than the interior diameter of a component of a delivery device such that valve 100 may be translated through slotted funnel 190 in the loading direction and transition smoothly from the funnel into the delivery device. In other words, when valve 100 is translated through slotted funnel 190 from entry end 191 to exit end 192, the valve may substantially abut the interior surface of the funnel, and the relative sizing of the exit end of the funnel and the opening of the delivery device receiving the valve may allow the valve to transition smoothly into the delivery device, the valve collapsing as it translates through the funnel.

Slotted funnel 190 includes slots 194 extending along a length of the funnel. Slotted funnel 190 has a wall thickness, and slots 194 may extend through the entire wall thickness of the funnel as illustrated in FIG. 5, or alternatively through part of the wall thickness of the funnel, so long as the slots are sufficiently deep that the free ends of the tines 107 of valve 100 do not contact and/or scrape the interior of the funnel as the valve translates through the funnel, as described further below. Slotted funnel 190 includes a plurality of slots 194 disposed along a length of the funnel and spaced approximately equal distances apart from one another. In some examples, slotted funnel 190 may include 24 slots and valve 100 may include 24 tines 107. Each slot 194 is a long and narrow partial or complete void defined by slotted funnel 190 configured to receive a corresponding tine 107 of valve 100. Each slot 194 extends from the circumference of exit end 192 of slotted funnel 190 to a circumferential axis near (but preferably spaced apart from) the circumference of entry end 191 of the funnel. In other words, slots 194 may not extend up to and through entry end 191 of slotted funnel 190, but may stop short before reaching the entry end, leaving a portion of the funnel as a solid continuous portion around the circumference at the entry end. Slots 194 may extend up to and through exit end 192, but need not extend through the entire thickness of slotted funnel 190 at the exit end, leaving a continuous portion of the circumference at the exit end similar to that of entry end 191. It is also contemplated that slots 194 may extend through the entire thickness of slotted funnel 190 up to and through exit end 192. Slots 194 have a first width near entry end 191 and a second width near exit end 192, the first width larger than the second width. The width of slots 194 tapers as the slots extend along the length of the slotted funnel 190 from entry end 191 to exit end 192 to allow for self-alignment of replacement valve 100 by guiding tines 107 in place as the valve translates through the funnel and maintains the radial alignment of the valve. Slotted funnel 190 further includes grooves 195 which further contribute to maintaining the alignment of valve 100. For example, stent cells of the atrial and/or ventricular anchors 102, 104 may be disposed at least partially within grooves 195 as valve 100 translates through slotted funnel 190, which may serve as an additional measure for preventing the valve from rotating out of alignment.

As noted above and shown in FIG. 2A, valve 100 includes tines 107 extending generally radially outward from anchor assembly 101 of the valve. Tines 107 are spaced approximately equal distances apart around the outer circumference of anchor assembly 101, which distance may correspond to (e.g., be equal to) the spacing between adjacent slots 194, at least near the entry end 191 of the slotted funnel. Tines 107 have generally traumatic tips that have the potential to scrape along the interior surface of slotted funnel 190 as valve 100 translates through the funnel. To accommodate for the traumatic tips of tines 107, each tine may align with a slot 194 of slotted funnel 190. As valve 100 translates through slotted funnel 190, each tine 107 may protrude into and/or through a corresponding slot 194, while the funnel may contact other portions of the valve and apply an inward force against the surface area of the valve surrounding the tines. In other words, valve 100 may be gradually compressed as it is translated through slotted funnel 190 while tines 107 (and particularly the traumatic free ends of the tines) generally do not contact any solid surface of the funnel. Such an arrangement may help to avoid scraping of tines 107 against slotted funnel 190 which might otherwise lead to particulate generation and/or wear of both the tines and the funnel. Further, the evenly spaced structure of tines 107 and the corresponding evenly spaced structure of slots 194 may promote an even or uniform collapse of valve 100 by forcing radial symmetry of the valve as the valve translates through the slotted funnel and the narrowing internal diameter of the funnel collapses the valve 100. That is, the nature of passing tines 107 through slots 194 maintains equal positioning of the tines with respect to each other as valve 100 is collapsed, causing a uniform collapse of the valve. This uniform collapse may be further supported via the interaction of stent cells with grooves 195.

Securement members 196 extend radially outward from the circumference of slotted funnel 190 at entry end 191. Slotted funnel 190 includes four securement members 196 spaced approximately 90 degrees apart from one another along the circumference, but any number of securement members spaced any distance apart is contemplated. Securement members 196 are similar to locking tabs 144 of ring 140 described above, the securement members adapted to secure slotted funnel 190 to either ring 140 or valve support 160. That is, securement members 196 include a lip 197 along the radially outermost portion of the securement members which includes a groove (not shown) on the proximal face of the securement members substantially similar to groove 147 on locking tab 144. Thus, in examples of packaging assembly 50 excluding ring 140, slotted funnel 190 may be coupled and locked to valve support 160 in the same or substantially the same manner as the ring is coupled to the valve support above, wherein protrusions 179 of the valve support engage the grooves on the securement members in the locked configuration. It should be noted that overhang 159 of secondary extension 156 on ring 140 includes a protrusion (not shown) substantially similar to protrusion 179, and thus in examples of packaging assembly 50 including the ring, the ring may be coupled to valve support 160 in the manner described above, and slotted funnel 190 may be coupled to the ring in substantially the same manner with the protrusions of the overhangs of the ring engaging the grooves of the securement members in the locked configuration. In other words, the similarity in structure of the connection mechanisms of valve support 160, ring 140 and slotted funnel 190 allows the funnel to be interchangeably coupled either directly to the valve support or to the ring which is coupled to the valve support. For purposes of clarity, in examples such as the embodiment shown in FIG. 1A, packaging assembly 50 may be referred to as being in a locked configuration when ring 140 is coupled to valve support 160, and slotted funnel 190 is coupled to the ring. Packaging assembly 50 may further be referred to as being in a partially locked configuration when two of the above components are coupled to each other, but the third component is not coupled to the other two, e.g., valve support 160 is coupled to ring 140 while ring is not coupled to slotted funnel 190, or the funnel is coupled to the ring while the ring is not coupled to the valve support. Still further, packaging assembly 50 may be referred to as being in an unlocked configuration when none of valve support 160, ring 140 nor slotted funnel 190 are coupled to one another. Similar to locking tabs 144, a radially outermost surface of securement members 196 includes ridges 198 which may facilitate a user in readily gripping the securement members to manipulate (e.g. rotating) slotted funnel 190. It should be understood that additional or alternative texturizations may be provided to assist with manipulation and/or gripping of portions of slotted funnel 190.

FIGS. 6A-6C show packaging assembly 50 according to an aspect of the present disclosure in a partially assembled configuration. It should be noted that packaging assembly 50 is shown in FIGS. 6A-6C without a slotted funnel 190 and without secondary extensions 156 coupled to primary extensions 149 of ring 140, and also shown in FIG. 6C without replacement heart valve 100, but otherwise includes the same structure as the embodiment described above. In this regard, packaging assembly 50 is designed for use in conjunction with replacement heart valve 100 to form a system for heart valve repair. Packaging assembly 50 includes ring 140 coupled to and positioned at least partially within valve support 160, as shown in FIGS. 6A and 6B. Ring 140 and valve support 160 may each be formed via injection molding, although other manufacturing methods may be suitable. Packaging assembly 50 further includes retention mechanism 180 (described further below) for securely attaching and tensioning replacement heart valve 100 so that the replacement heart valve is already prepared for attachment to the delivery device while in the packaging, allowing for more efficient implantation. Assembled ring 140 has an inner diameter sized such that the ring fits snugly around central portion 103 of replacement heart valve 100 to prevent the replacement heart valve from shifting when positioned in a final configuration in the packaging assembly 50. In some embodiments, the inner diameter of ring 140 is about equal to, slightly larger, or slightly smaller than, the exterior diameter of central portion 103 of replacement heart valve 100 while the replacement heart valve is in the expanded condition. Accordingly, ring 140 has an outer diameter that allows the ring to fit within the valve support.

Ring 140 may attach to valve support 160 with an attachment mechanism that allows the ring to removably attach to the valve support such that the ring and valve support can be moved from an unlocked configuration in which the two are detached and can rotate relative to one another to a locked configuration in which the ring is rotationally and axially locked to the valve support. Generally, valve support 160 forms the holding component of the packaging assembly 50 in which replacement heart valve 100 is securely positioned. Accordingly, valve support 160 defines an inner diameter that allows the ring to fit within the valve support. Further, the height and outer diameter of valve support 160 are sized and configured to fit within a shipment jar or packaging container. With this sizing, valve support 160 does not interfere with the seal of the jar or container. Additionally, the size and shape of valve support 160 allows for easy insertion and removal of the valve support from the jar or container, such that sufficient clearance is provided to allow for insertion and removal of the valve support without damage to the valve support and/or replacement heart valve 100, while also partially or fully limiting motion of the valve support within the jar or container during transportation.

As shown in FIG. 6B, retention mechanism 180 of packaging assembly 50 includes pin 182, cap 186 for attaching to pin 182, and sutures (or other thread-like components) 188 for attaching the retention mechanism to replacement heart valve 100. A first end of each of sutures 188 is secured to cap 186 and a second end of each suture includes a loop to slip over and attach to attachment members 118 of replacement heart valve 100. Retention mechanism 180 allows for secure attachment of replacement heart valve 100 within packaging assembly 50 during shipment, removal from the shipment jar or container, rinsing of the replacement heart valve, and coupling to the delivery device.

As shown in FIG. 6C, cannulated pin 182 includes a distal portion 183 having a first diameter which transitions to a proximal portion 184 having a second diameter at step 185, the second diameter being smaller than the first diameter. Proximal portion 184 is sized and configured to fit within an inner lumen (not shown) of cap 186. Cap 186 can be attached to pin 182 via a cooperating threaded engagement between an external surface of proximal portion 184 and an inner surface of cap 186 (not shown) which defines the lumen.

Step 185 forms a shoulder to control the position of cap 186 on pin 182. The outer surface of cap 186 is threaded for attaching the delivery system to the retention mechanism 180 of packaging assembly 50. Cap 186 includes a portion, either monolithic with the cap or attached thereto, that enables sutures 188 to be threaded therethrough. In the illustrated embodiment, cap 186 includes collar 187 having a plurality of openings (not shown) for receiving suture strands 188 such that a first end of the suture can be secured through the openings thereby securing sutures 188 to the cap. Cap 186 and collar 187 may be monolithic, e.g. constructed from a single piece, or they may be separate pieces mechanically joined together thereafter. A second end of each suture 188 is looped over a respective one of the plurality of attachment members 118 on a proximal end of replacement heart valve 100, creating a connection between the valve and the pin. Sutures 188 may be tensioned, e.g., by maintaining replacement heart valve 100 at a distance relative to pin 182. In one example, there may be twelve suture strands 188 connected to pin 182 and attachment members 118. In order to achieve tension on suture strands 188, pin 182 is designed to have a height sufficient to create such tension when the suture strands are positioned through the retaining elements and cap 186. Pin 182 can be positioned within rod 178 to the desired height based on the size of the replacement heart valve. In order to maintain pin 182 within the lumen of rod 178, the pin is structured to facilitate an interference fit. In one example, pin 182 may be tapered such that the outer diameter of the pin at a particular location is greater than the inner diameter of rod 178 at a particular location. This enables pin 182 to be inserted to a specified depth and kept in place. Alternatively, pin 182 could include threads for threaded engagement with the internally threaded rod, or the pin and/or lumen of the rod could be stepped and the engagement could be achieved through a friction fit of the stepped configuration. Crimp members 189, such as knots, may be provided around at least a portion of each suture strand 188 to form the loop at the end of the suture. Crimp members 189 may also help to prevent possible hooking onto other features of the packaging assembly. In some cases, the crimps or coils on the sutures are secured in place by an adhesive, which can help maintain tension and avoid inadvertent separation of the sutures. Although described herein as suture, the material can alternatively be wire or another flexible member capable of tensioning the replacement heart valve 100.

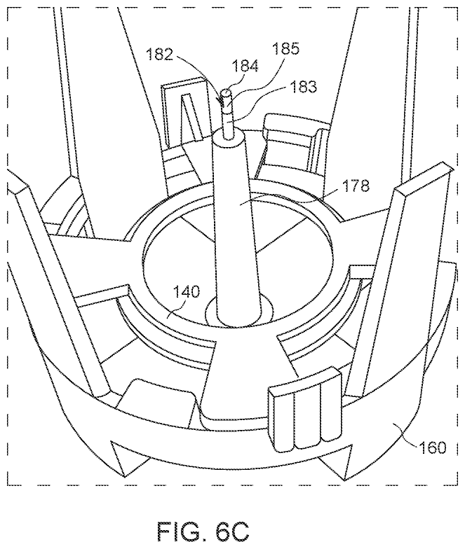

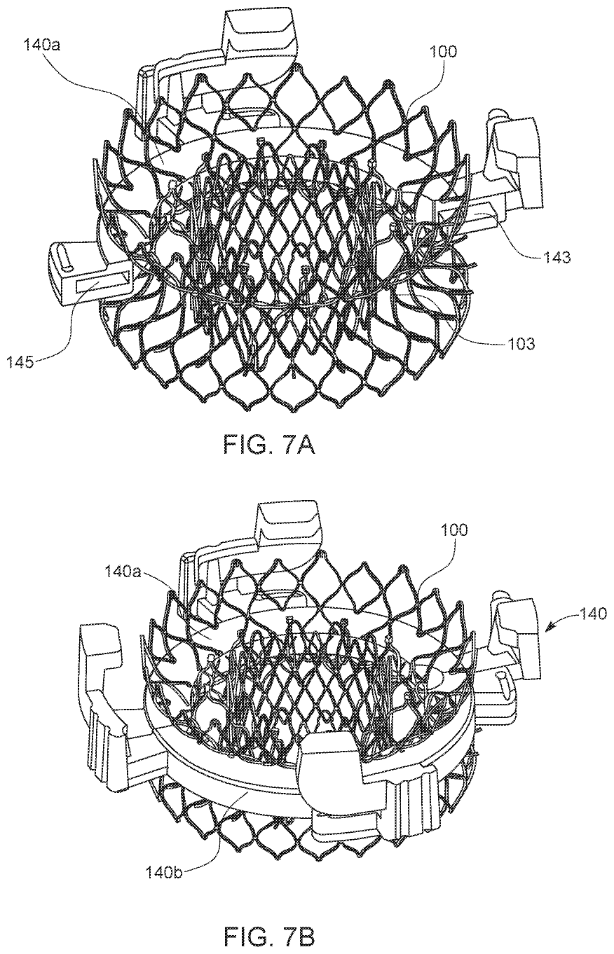

Ring 140 may be assembled with valve 100, as shown in FIGS. 7A-7B. As described above, ring 140 may be modular such that it may be separated into two (or more) modular pieces, first ring portion 140a and second ring portion 140b, which may be detachably coupled to one another. First ring portion 140a may be positioned around anchor assembly 101 of valve 100 such that the first ring portion circumferentially surrounds about half of the central portion 103 of the valve as shown in FIG. 7A. Ring 140 may be sized such that the inner diameter of the ring (i.e., the distance between opposite points along the inner circumference of the ring) is substantially similar to the diameter of central portion 103 of valve 100 and smaller than the diameter of the valve at the atrial and ventricular anchors when the ring is assembled and the valve is mostly or fully expanded. Such relative sizing between ring 140 and valve 100 allows the ring to be nested within central portion 103 of the valve to securely hold the valve in place relative to the packaging assembly, thereby preventing undesired movement (including axial movement) of the valve when in the packaged configuration during shipping. Second ring portion 140b may be positioned around the remaining uncovered portion of central portion 103 of valve 100 in a manner substantially similar to that of first ring portion 140a, and protrusions 143 of each of first and second ring portions 140a, 140b mate with the corresponding apertures 145 of the other ring portion to detachably couple the first and second ring portions together to form assembled ring 140. This configuration may allow for the ring 140 to surround the valve 100 without having to crimp the valve and pull the valve through the open center of the ring, which might otherwise be required if the ring were formed as a single monolithic piece.

As noted above, packaging assembly 50 is shown in a partially assembled configuration in FIGS. 6A-6C, in which ring 140 and valve support 160 are locked together and replacement heart valve 100 is positioned within and secured to ring 140, the ring is positioned within valve support 160 and rotated in a first direction such that locking tabs 144 slide into the grooves defined by ledges 173 and mechanically lock to valve support 160 via the snap fit connection. Pin 182 is positioned within a proximal portion of cannulated rod 178 with proximal portion 184 of the pin extending beyond the rod. Body 141 of ring 140 is positioned so that it securely fits around the waist defined by central portion 103 of the hour-glass shaped anchor assembly 101 of replacement heart valve 100. Replacement heart valve 100 is positioned with ventricular anchor 104 adjacent base 166 of valve support 160. This allows for a circumferentially snug and secure fit which reduces and/or prevents movement of replacement heart valve 100 within packaging assembly 50. Pin 182 extends coaxial with a longitudinal axis of replacement heart valve 100. Cap 186 is attached to pin 182 with suture strands 188 looped through the openings of cap 186. For ease of assembly, suture strands 188 can be looped through the openings of cap 186 prior to attachment of the cap with valve support 160 and ring 140 structure. The loops of suture strands 188 are attached to attachment members 118 of atrial anchor 102 of replacement heart valve 100 thereby tensioning suture strands 188. The tension on suture strands 188 prevents inadvertent release of the suture loops on attachment members 118 (e.g. by preventing the suture loops from slipping off the pins of the attachment members 118). The tension on the suture strands 188, in combination with ring 140 limiting axial movement of the prosthetic valve 100, also constrains the valve fully in the axial direction. Further, the tension of suture strands 188 also holds cap 186 against the shoulder of pin 182. Thus, the positioning of body 141 of ring 140 around the valve together with the tension of suture strands 188 provides for full axial and circumferential securement of replacement heart valve 100. In the process of loading valve 100 into a delivery device, once the valve is connected to the delivery device, the tension is transferred from ring 140 to the delivery device, and the ring no longer serves a function and may thus be disassembled by separating first ring portion 140a from second ring portion 140b to remove the ring from the assembly altogether.

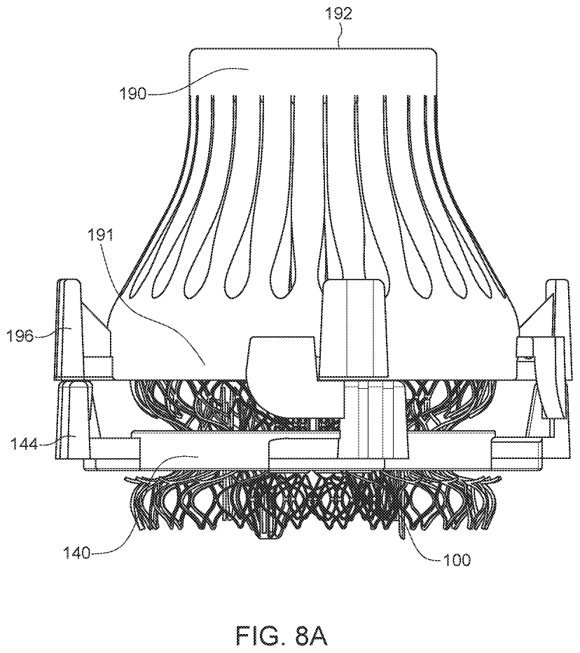

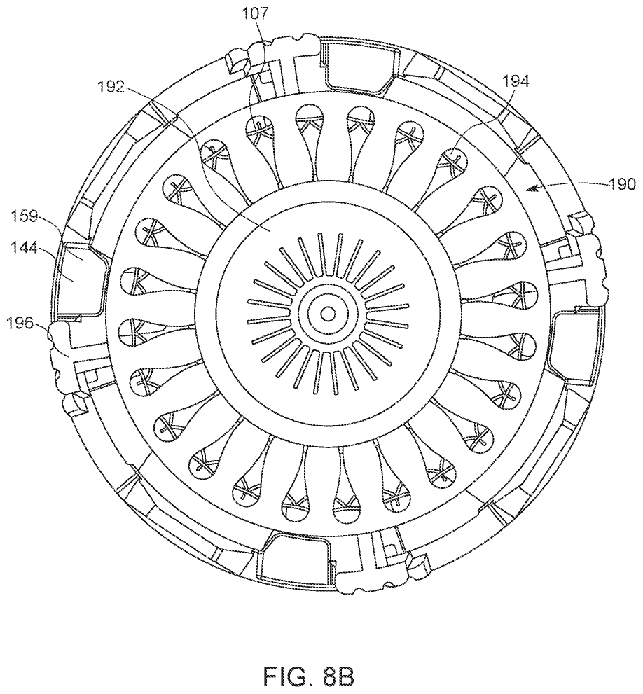

An assembled ring 140 surrounding valve 100 is shown in FIG. 8A with slotted funnel 190 coupled to the ring. Slotted funnel 190 is oriented with entry end 191 adjacent ring 140. Each securement member 196 on slotted funnel 190 is coupled to a corresponding locking tab 144 on ring 140. To couple securement members 196 to locking tabs 144, entry end 191 of slotted funnel 190 may be positioned adjacent ring 140 such that a distal surface of the securement members (i.e., surface facing downward in FIG. 8A) abuts a proximal surface of the locking tabs (i.e., surface facing upward in FIG. 8A), and the funnel may then be rotated in a locking direction relative to the ring so that the lip 197 of each securement member engages with and slides under the overhang 159 of each locking tab, and a protrusion on the overhang engages with a groove on the lip as described above, inhibiting any relative rotational or axial movement between the funnel and the ring. The engagement between securement members 196 and locking tabs 144 may generally maintain the ring and slotted funnel in their respective positions until a user manipulates the assembly to rotate the funnel in an opposite, unlocking direction relative to the ring to release the funnel from the locked position.

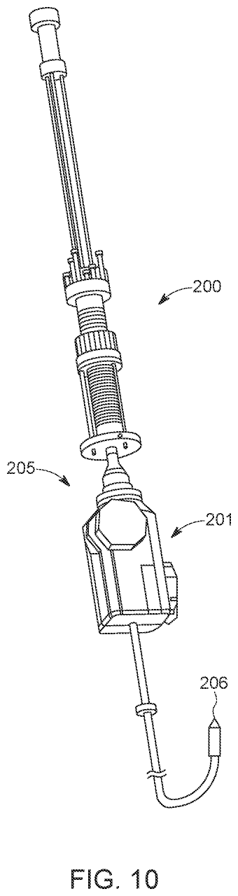

FIG. 8B illustrates a proximal end view of the assembly shown in FIG. 8A showing exit end 192 of slotted funnel 190. Each locking tab 144 of ring 140 is shown engaged with a corresponding securement member 196 of slotted funnel 190, in which lip 197 of each securement member is covered by overhang 159 the corresponding locking tab in the illustrated view. Further, each tine 107 of valve 100 is shown in alignment with a corresponding slot 194 of slotted funnel 190 to encourage and maintain radial alignment of the valve relative to the funnel. Exit end 192 of slotted funnel 190 may include a cylindrical indentation wherein the indented portion of the exit end has a first radius which is smaller than the radius of packaging assembly 50 overall. That is, exit end 192 includes a circumferential portion which is not indented and extends farther in the proximal direction than the cylindrically indented portion. The cylindrical indentation may be sized and shaped to receive delivery device 60, as shown in FIGS. 10E and 10F, so that the delivery device can securely engage with exit end 192 of slotted funnel 190 and allow for a smooth transition of replacement heart valve 100 from the funnel to the delivery device. Further, it should be noted that each slot 194 shown in FIG. 8B on exit end 192 of funnel 190 may be a continuation of the corresponding slot extending along the side wall of the funnel, which allows tines 107 to be translated completely through the funnel without interruption.

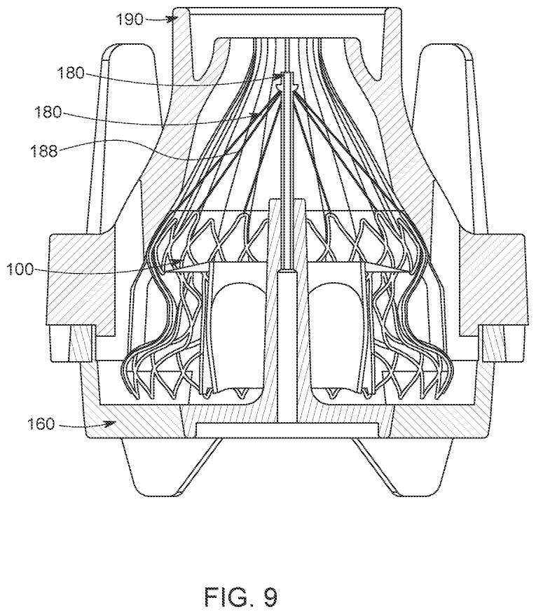

In some examples, as shown in FIG. 9, packaging assembly 50 need not include ring 140. The example of FIG. 9 includes valve support 160, valve 100 coupled to the valve support, and slotted funnel 190 coupled directly to the valve support and positioned over the valve. In such an example, securement members 196 of slotted funnel 190 couple to groove 177, ledge 173 and protrusion 179 of valve support 160 as described above. The inner surface of slotted funnel 190 may apply a force to valve 100 resisting the force caused by the tension applied by sutures 188. In other words, sutures 188 may be coupled to attachment members 118 of valve 100, and the valve may abut the inner surface of slotted funnel 190 to create and maintain the tension in the sutures. Thus, in the example without ring 140, valve 100 may remain under substantially the same forces applied by sutures 188 and slotted funnel 190 to hold the valve securely in place within the packaging assembly during shipping.

The loading of replacement heart valve 100 from packaging assembly 50 into a delivery device 200 will be described herein. It should be noted that with regard to the loading process described, replacement heart valve 100 may be loaded into any delivery device, and is not limited to the delivery device described herein. It should also be noted that delivery device 200 may be suited to receive and deploy any replacement heart valve, and is not necessarily limited to replacement heart valve 100 disposed in packaging assembly 50 as described above.

Delivery device 200 is shown in FIG. 10 extending from a proximal region 205 to a distal end 206, wherein the proximal region is relatively nearer the user (and farther away from the patient) and the distal end is farther from the user (and may also be considered the “leading end” of the device that first enters the patient), when the delivery device 200 is being used as intended. As described below in greater detail, proximal region 205 may include a base unit and various controls for a user to manipulate and maneuver the distal end of delivery device. Extending from the base unit is a plurality of catheters disposed within an outer catheter, each catheter adapted to be controlled at the base unit. Several components described below and shown in the figures may be detachably coupled to delivery device 200, e.g., for the loading procedure, but may nonetheless be referred to as a component of the delivery device.

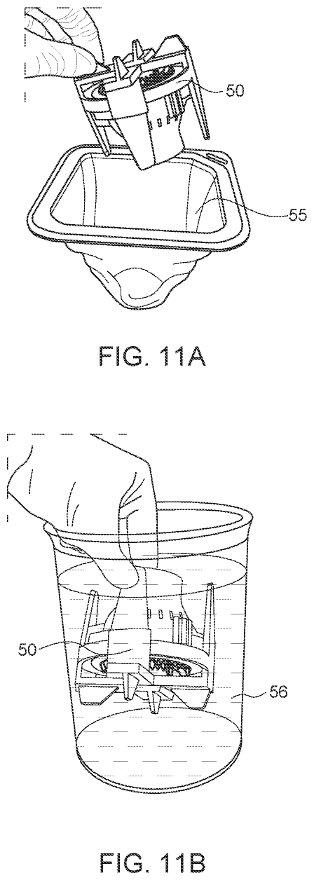

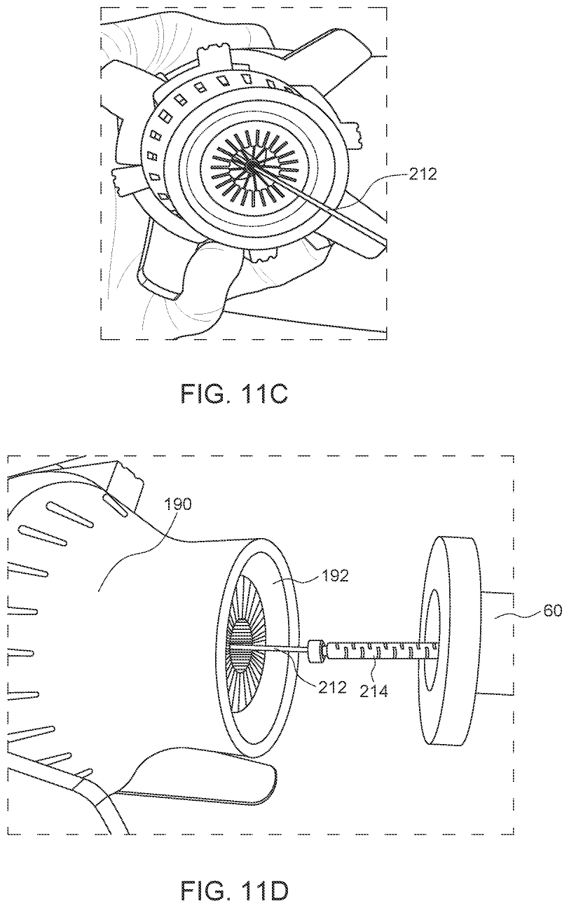

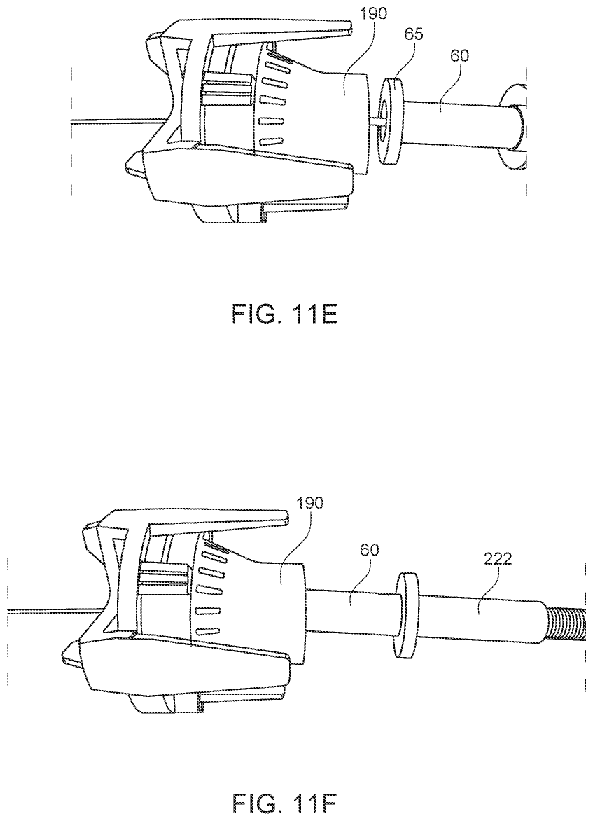

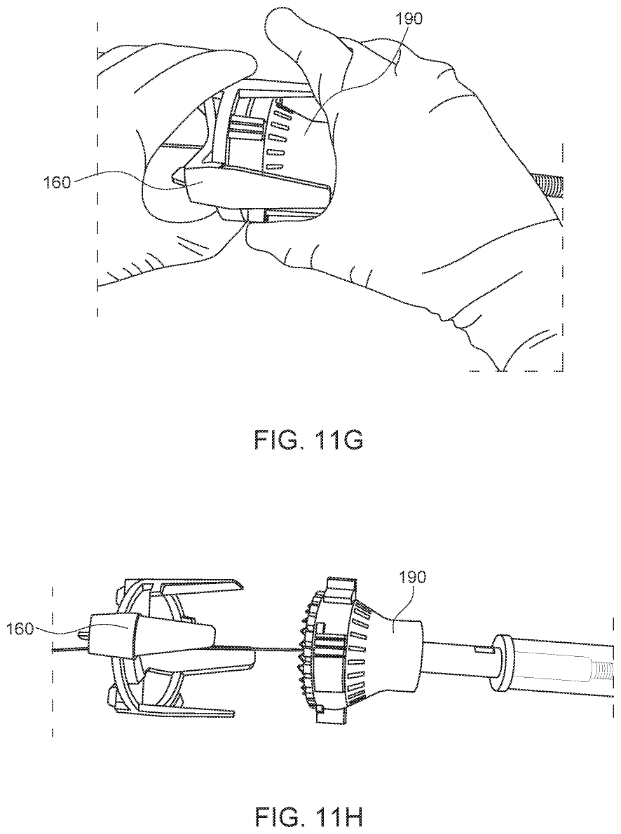

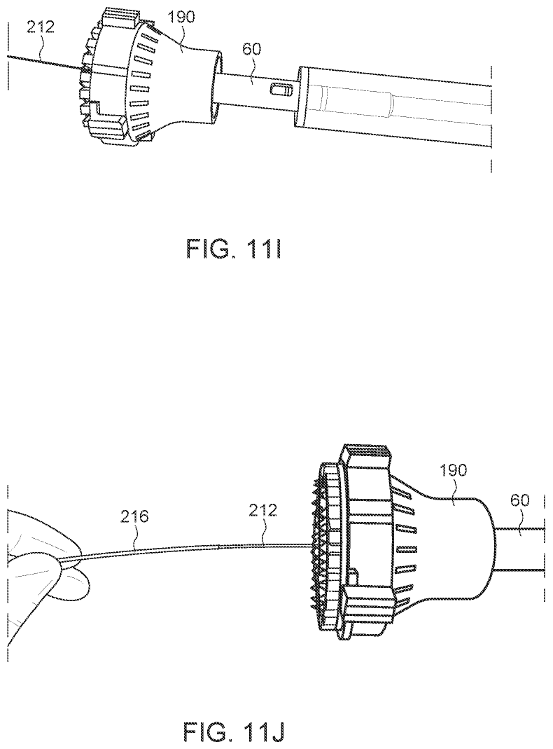

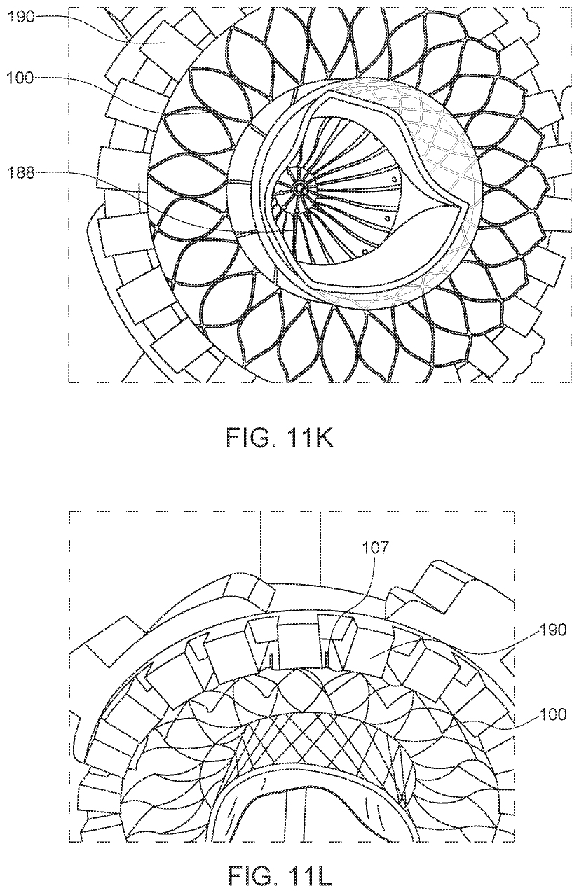

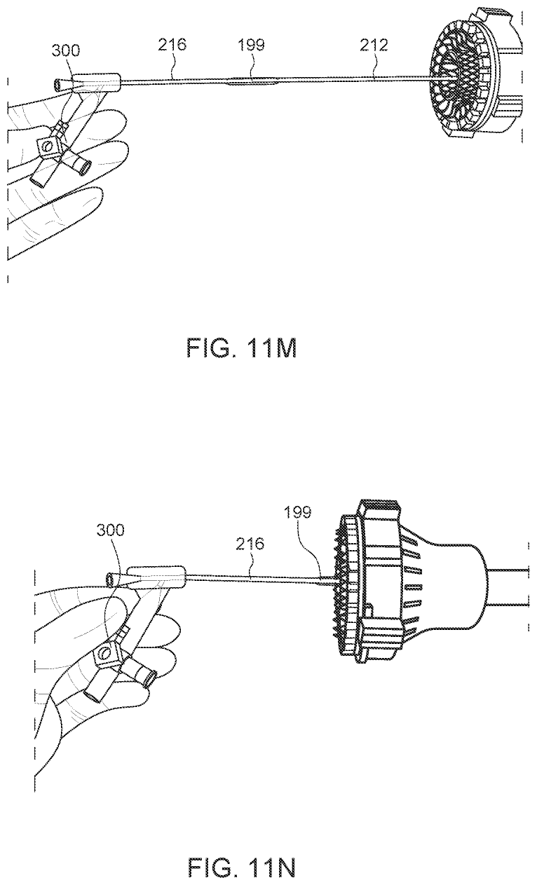

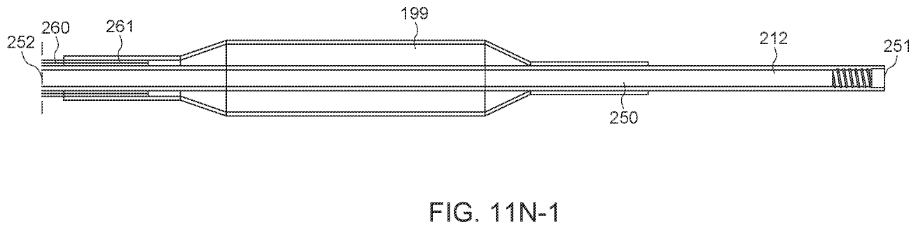

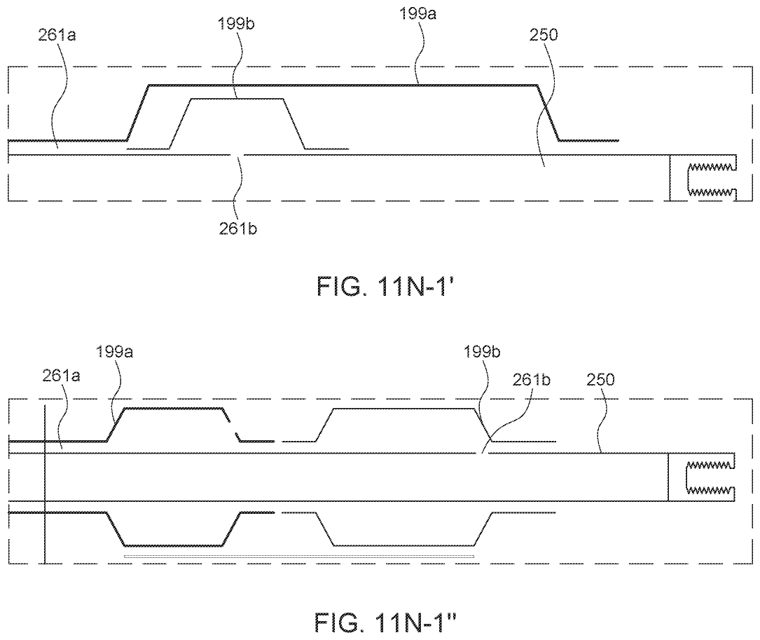

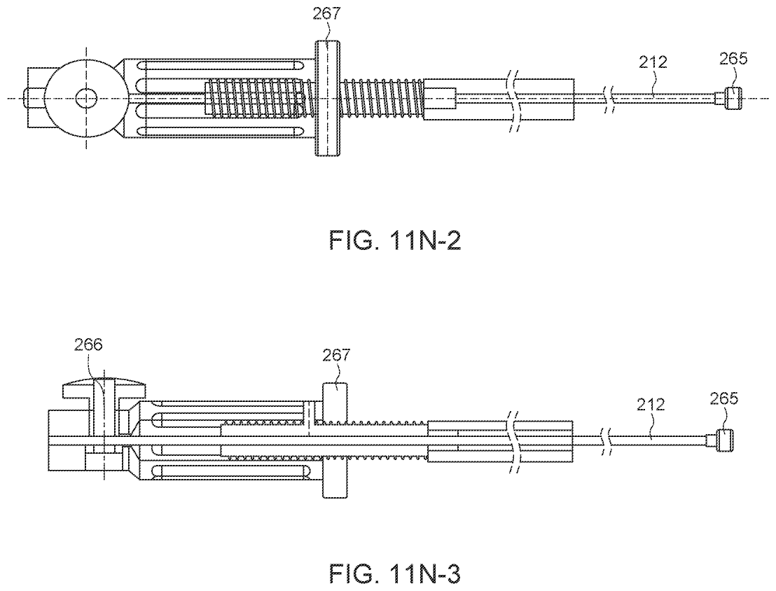

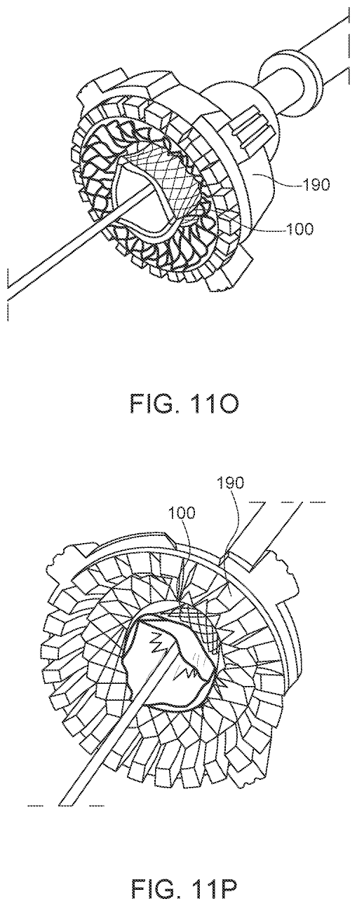

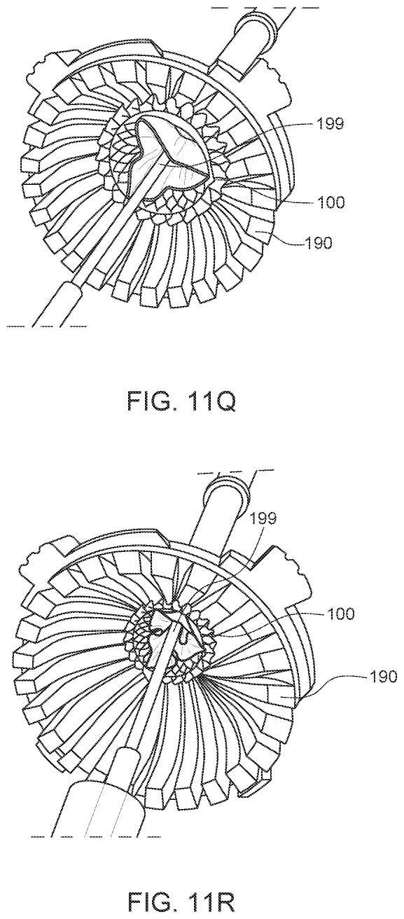

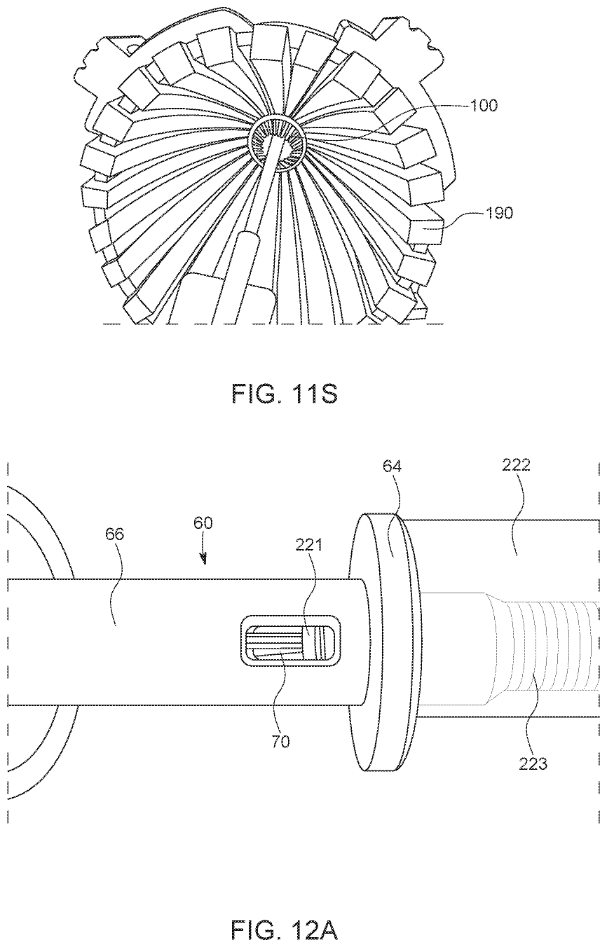

FIGS. 11A-11S illustrate various steps of removing a fully assembled packaging assembly 50 from its packaging and transferring replacement valve 100 from the packaging assembly to a delivery device according to embodiments of the disclosure. An operator or user, such as a surgeon, may remove packaging assembly 50 from packaging 55 by holding onto at least one rib 171 as shown in FIG. 11A, and may submerge the packaging assembly into a liquid such as saline 56 to rinse or wet the assembly, as shown in FIG. 11B. It should be understood that packaging 55 may include a seal, cap, or other member that must be removed prior to removing packaging assembly 50 from packaging 55.

A mandrel 212 extending from delivery device 200 may be inserted into a proximal end of packaging assembly 50, passed through exit end 192 of slotted funnel 190, and further passed through rod 178 and pin 182 as shown in FIG. 11C. Mandrel 212 may help to align retention mechanism 180 with a second funnel or window funnel 60 (described below) to maintain the coaxial nature of cap 186 and the lumen of the window funnel when replacement heart valve 100 is being connected to delivery device 200. Mandrel 212 may be coupled at the proximal end of the delivery device (e.g., at a handle or base unit of the delivery device), extend distally through an entire length of the delivery device, and as shown in FIG. 11D, extend distally from an interior lumen of a suture catheter 214. Suture catheter 214 may extend from an interior lumen of window funnel 60, and the suture catheter may be inserted through exit end 192 of slotted funnel 190 to couple to retention mechanism 180, while using mandrel 212 for ease of alignment. In other words, suture catheter 214 may ride over mandrel 212 until a distal tip of the suture catheter abuts or contacts cap 186. Packaging assembly 50 may then be threadably attached to delivery device 200 by threading the distal tip of suture catheter 214 over cap 186 of retention mechanism 180. Exemplary methods of attaching the delivery system to the replacement heart valve are described in U.S. Patent Publication No. 2018/0092744, the disclosure of which is hereby incorporated by reference herein. After suture catheter 214 is coupled to retention mechanism 180, the suture catheter may be retracted slightly in the proximal direction to apply at least a minor tension to the sutures to keep them securely coupled to attachment members 118. Suture catheter 214 may be formed by a spiral laser cut in a metal so that the catheter is at least partially flexible. The distal tip having an internal threading to couple to retention mechanism may be laser welded to the tube portion of the suture catheter.

Window funnel 60 is positioned at or near the distal end of delivery device 200 between a loading tube 222 and slotted funnel 190. That is, at its distal end, window funnel 60 is detachably coupled to slotted funnel 190, and at its proximal end, the window funnel is detachably coupled to valve cover 223 and/or loading tube 222 (shown in FIG. 11F). While window funnel 60 is shown to have a generally cylindrical exterior surface, the window funnel may have an interior diameter that tapers in the loading direction (i.e., from the distal end to the proximal end) to continue forcing replacement heart valve 100 further into the collapsed condition. Window funnel 60 may have an interior diameter at its distal end suitable for receiving replacement heart valve 100 from exit end 192 of slotted funnel 190 such that the valve can transition smoothly from the slotted funnel into the window funnel. Similarly, window funnel 60 may have an interior diameter at its proximal end suitable for transitioning replacement heart valve 100 smoothly from the window funnel into a can 221 and valve cover 223 of delivery device 200 (described further below).

After coupling suture catheter 214 to retention mechanism 180, the suture catheter may be retracted proximally (i.e., toward delivery device 200) as shown in FIG. 11E, and slotted funnel 190 may be positioned over window funnel 60 as shown in FIG. 11F so that the slotted funnel is fixed relative to the window funnel during loading of the valve into the delivery device. As described above, window funnel 60 may engage with packaging assembly 50 such that the window funnel is inserted into the cylindrical indentation of slotted funnel 190 and a distal flange 65 of the window funnel abuts the corresponding cylindrical indentation of the slotted funnel. Further, lumen 193 of slotted funnel 190 at exit end 192 is aligned with a lumen of window funnel 60 to allow for a smooth transition of valve 100 from the slotted funnel to the window funnel. Once slotted funnel 190 is positioned to abut window funnel 60, suture catheter 214 may be further retracted proximally to pull cap 186 and sutures 188 of retention mechanism 180 and cause valve 100 to apply a degree of pressure onto the inner surface of the slotted funnel. Valve support 160 may be decoupled from slotted funnel 190 in the manner described above. That is, the user may rotate valve support 160 relative to slotted funnel 190 in a direction to overcome the snap fit and transition the valve support from a locked configuration to an unlocked configuration, as shown in FIGS. 11G and 11H. Valve support 160 may be removed distally, e.g., in a direction away from the window funnel 60. It should be noted that valve support 160 may be decoupled from slotted funnel 190 before or after valve 100 has been translated through the slotted funnel and loaded into window funnel 60. It should also be noted that in examples of packaging assembly 50 including ring 140, the ring may be disassembled and removed from the packaging assembly at any time after suture catheter 214 is coupled to retention mechanism 180 and retracted to cause replacement heart valve 100 to contact and apply a minimal degree of force against slotted funnel 190.

After components of packaging assembly 50 (e.g., valve support 160, ring 140) are removed so that only valve 100 remains inside slotted funnel 190 coupled to window funnel 60 as shown in FIG. 111, a nosecone catheter 216 may be applied over mandrel 212, e.g., by the user. It is contemplated the nosecone catheter 216 may be stored within suture catheter 214 and may be translated distally relative to mandrel 212 after packaging assembly 50 is removed. Nosecone catheter 216 is a hollow tube having an opening at its proximal end so that the proximal end of the nosecone catheter is adapted to slide over the distal end of mandrel 212, and/or mandrel is adapted to slide through the nosecone catheter (in some examples, the mandrel may be passed proximally through the nosecone catheter). Nosecone catheter 216 has an internal diameter substantially the same as or slightly larger than an outer diameter of mandrel 212, so that the nosecone catheter may be advanced proximally relative to the mandrel so that at least a portion of the mandrel is disposed within the nosecone catheter, as shown in FIG. 11J. Nosecone catheter 216 has an external diameter substantially the same as or slightly smaller than an internal diameter of suture catheter 214, so that as the nosecone catheter is advanced over mandrel 212, the nosecone catheter may be advanced internally through the suture catheter such that the nosecone catheter is at least partially positioned inside (e.g., radially inward of) the suture catheter.

After nosecone catheter 216 is placed over mandrel 212 (or the mandrel is passed proximally through the nosecone catheter), suture catheter 214 may then be further retracted proximally into delivery device 200 to pull valve 100 through slotted funnel 190 via sutures 188 as shown in FIG. 11K. Valve 100 may be oriented such that each tine 107 aligns with a corresponding slot 194 in slotted funnel 190, as shown in FIG. 11L, and the radial alignment of the valve may be maintained by the slots and grooves 177 on the inner surface of the slotted funnel as the valve is translated therethrough. As described above, valve 100 is translated through the tapering diameter of slotted funnel 190, thereby transitioning the valve from the expanded condition to the collapsed condition (or substantially collapsed) upon reaching exit end 192 of the slotted funnel. In some examples, an internal support member may be used to assist in the uniform collapse of valve 100, as shown in FIGS. 11M-11S.

In some examples, the internal support member may be a balloon 199 coupled to a hypotube 250, which is illustrated in FIG. 11N-1. Hypotube 250 extends from a proximal end 251 to a distal end 252, and the hypotube may be threaded at its proximal end and may couple to a secondary tube 260 at its distal end. Hypotube 250 may be formed of a metal and secondary tube 260 may be formed of a polymer. Balloon 199 may extend around the exterior of hypotube 250 and longitudinally along a length of the hypotube. Balloon 199 may be adhered to hypotube 250 at a first end (e.g., the balloon's proximal end), and the balloon may be adhered to secondary tube 260 at a second end (e.g., the balloon's distal end). It is contemplated that balloon 199 may be coupled to hypotube 250 and secondary tube 260 by any suitable means, such as glue or an adhesive. Proximal end 251 of hypotube 250 may couple to a distal end of nosecone catheter 216, e.g., by a threaded connection, and the hypotube may define a lumen having a diameter sufficient to allow the hypotube to pass over mandrel 212. Thus, as nosecone catheter 216 is passed over and along mandrel 212 as shown in FIG. 11J, hypotube 250 may follow directly behind the nosecone catheter also passing over the mandrel. As shown in FIGS. 11M and 11N, a fluid fitting 300 (e.g. a “Luer-Lock” or a similar fitting) may be coupled to a distal end of secondary tube 260, the fluid fitting in communication with an internal space of balloon 199 through a pathway 261 shown in FIG. 11N-1. Pathway 261, positioned between the outer diameter of hypotube 250 and inner diameter of secondary tube 260, allows a user to inject and withdraw a fluid such as a saline solution into and from balloon 199 to inflate and deflate the balloon as desired during the loading process.

FIGS. 11N-1′ and 11N-1″ illustrate alternative examples of an internal support member coupled to hypotube 250. Shown in FIG. 11N-1′, the internal support member may include a first balloon 199a substantially similar to balloon 199 described above, and the internal support member may further include a second balloon 199b coupled to hypotube 250 and positioned internal to the first balloon. The presence of second balloon 199b inside first balloon 199a may allow an operator to balance and counteract forces applied to the first balloon. For instance, as valve 100 is translated through slotted funnel 190 and transitioned toward a collapsed condition, the forces applied by the valve to the balloon may be off-center or more concentrated on one end of the balloon than the other end. The unbalanced forces may deform first balloon 199a into an undesirable shape which may lead to an improperly collapsed valve 100. However, an operator may control the volume and pressure of first balloon 199a through a pathway 261a, and may also control the volume and pressure of second balloon 199b (e.g., by injecting or withdrawing saline through a pathway or access opening 261b independent from pathway 261a used to access first balloon 199a) to further adjust the pressure and volume of first balloon 199a to counteract and balance the unbalanced forces applied to the first balloon. The same concept may be applied to the embodiment shown in FIG. 11N-1″, however in this example, first balloon 199a and second balloon 199b are sequential and adjacent, rather than the second balloon positioned inside the first balloon. That is, second balloon 199b is positioned proximal to first balloon 199a. Similar to the embodiment described above, first balloon 199a may be accessed through pathway 261a, while second balloon 199b may be accessed through an independent pathway or opening 261b to separately control the pressure and volume of each balloon via saline injection and withdrawal. It is also contemplated that first and second balloons 199a, 199b may be surrounded by a third balloon that engulfs both the first and second balloons in a manner similar to the embodiment shown in FIG. 11N-1′.

In some examples, mandrel 212 may include a counterbearing 265 coupled to the distal tip of the mandrel, as shown in FIGS. 11N-2 and 11N-3. As noted above, mandrel 212 may extend through an entire length of the delivery device to reach the proximal end of the delivery device, for example a handle portion, where the delivery device is adapted to be operated by a user. For example, a clamp 266 may be included at the proximal end of the delivery device to clamp mandrel 212 and suture catheter 214 together so that they are translated in tandem. In some examples, the connection mechanism may be secured to mandrel 212 via a notch in the proximal end of the mandrel and a thumb button may translate such that a keyhole slot feature interlocks with the notch in the mandrel. Further, a knob 267 may be actuated (e.g., rotated) to tension the system and hold mandrel 212 in place relative to suture catheter 214. Counterbearing 265 may be coupled to the distal end of mandrel 212, for example, after nosecone catheter 216 and hypotube 250 are positioned over and surrounding the mandrel, as shown in FIG. 11J. With secondary tube 260 coupled to the distal end of hypotube 250 as described above, the secondary tube is positioned to abut counterbearing 265. Counterbearing 265 may also or alternatively abut a distal end of hypotube 250. Counterbearing 265 may be a disc, ball, or the like having a diameter larger than the internal diameter of the secondary tube 260 and hypotube 250. Thus, counterbearing 265, in combination with the proximal end of the mandrel 212 being clamped, prevents hypotube 250 from translating distally relative to mandrel 212, which is a notable feature for the loading process when valve 100 is collapsed around balloon 199, as described below in greater detail.

Returning to nosecone catheter 216, after hypotube 250 is securely attached thereto, the nosecone catheter may be retracted proximally into delivery device 200 until the nosecone catheter is fully seated within the delivery device such that balloon 199 is axially aligned with replacement heart valve 100. FIGS. 11O-11S show valve 100 in stages being translated through slotted funnel 190 and transitioning from the expanded condition to the substantially collapsed condition. As illustrated in FIG. 11S, valve 100 is in a substantially collapsed condition in which it is sized to pass through exit end 192 of slotted funnel 190. Suture catheter 214 may be further retracted until valve 100 has transitioned from positioning within slotted funnel 190 to positioning within window funnel 60.

As replacement heart valve 100 is translated proximally through slotted funnel 190 via suture catheter 214, the valve is axially aligned with and collapses over inflated balloon 199, which encourages a uniform collapse of the valve. Due, at least in part, to the tapering diameter of slotted funnel 190, a radially inward force from valve 100 is first applied to the proximal end of the balloon before the distal end of the balloon, and the force applied to the proximal end of the balloon may be of greater magnitude relative to the force applied to the distal end at a particular stage in the loading process. In any event, balloon 199 may be subject to unbalanced forces from valve 100. Such forces may tend to include a force on balloon 199 in the distal direction. Such force may be transferred from balloon 199 to hypotube 250 due to the adherence of the two elements. However, as noted above, secondary tube 260 or hypotube 250 is abutted by counterbearing 265 coupled (e.g., threadably, via a weld, adhesive, etc.) at the distal end of mandrel 212 and thereby counteracts forces applied to balloon 199 to generally hold the hypotube 250 and therefore the balloon in place relative to suture catheter 214 and valve 100 as the valve is collapsed within slotted funnel 190. In other words, as the replacement heart valve 100 collapses over the balloon 199, there is a tendency for the balloon to “want” to “jump” or rapidly translate distally relative to the replacement heart valve 100 due to the forces applied from the collapsing valve. However, counterbearing 265, and the clamping of the mandrel 212, reduces or eliminates the ability of the balloon 199 (and thus hypotube 250) to translate relative to the valve 100 during the loading process.

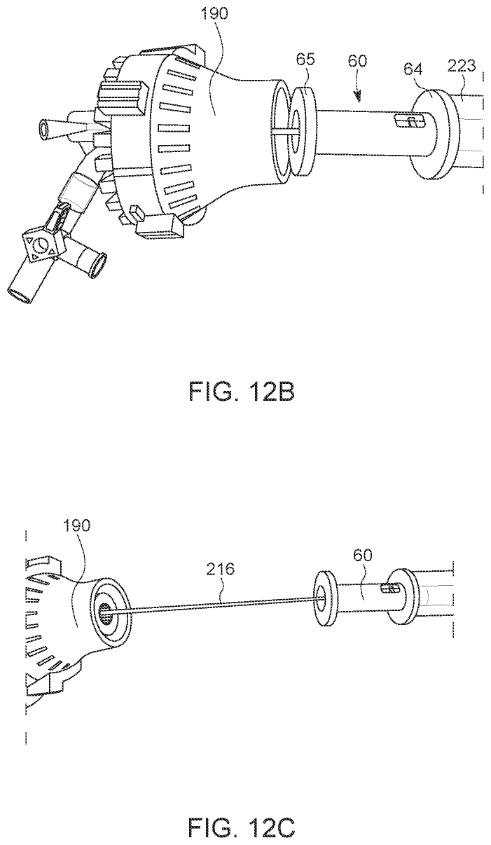

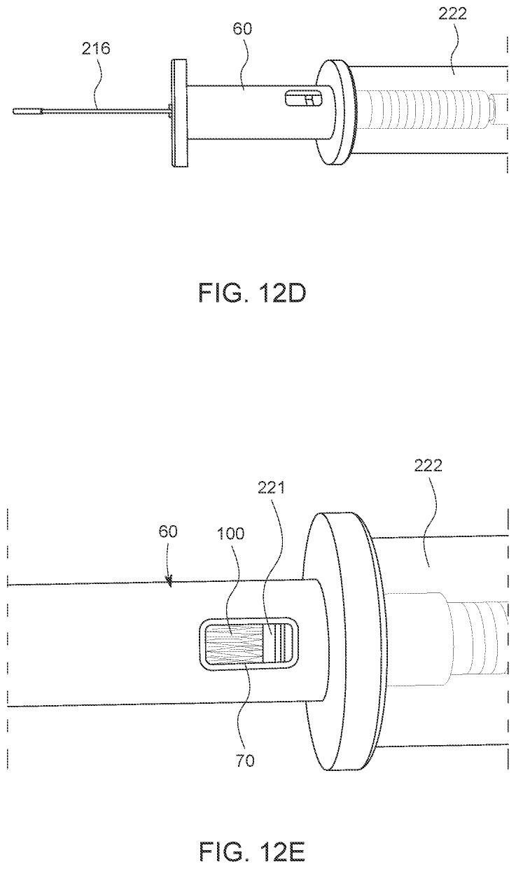

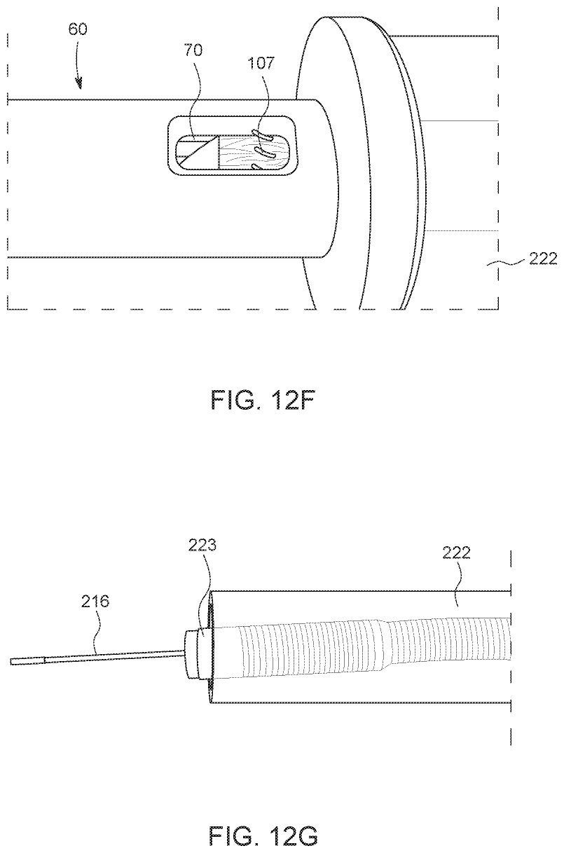

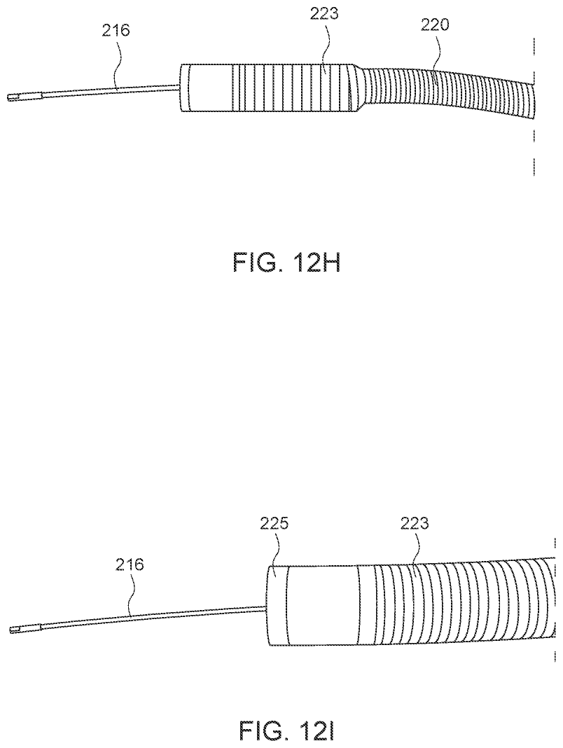

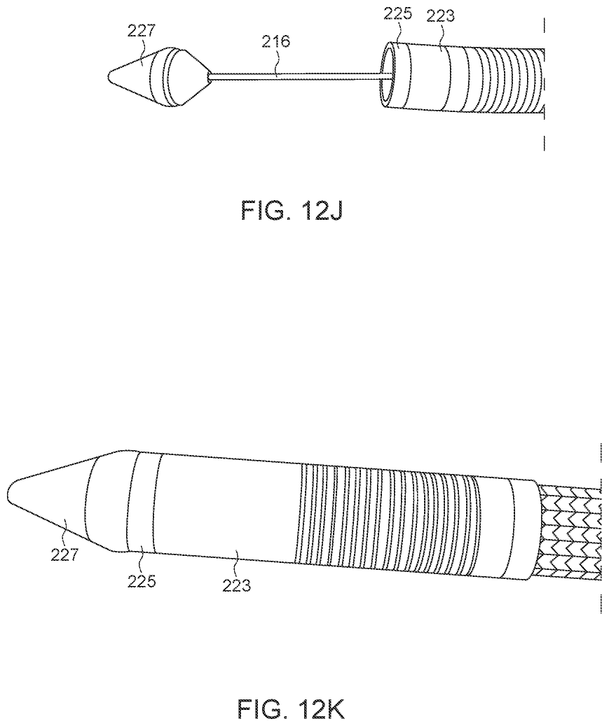

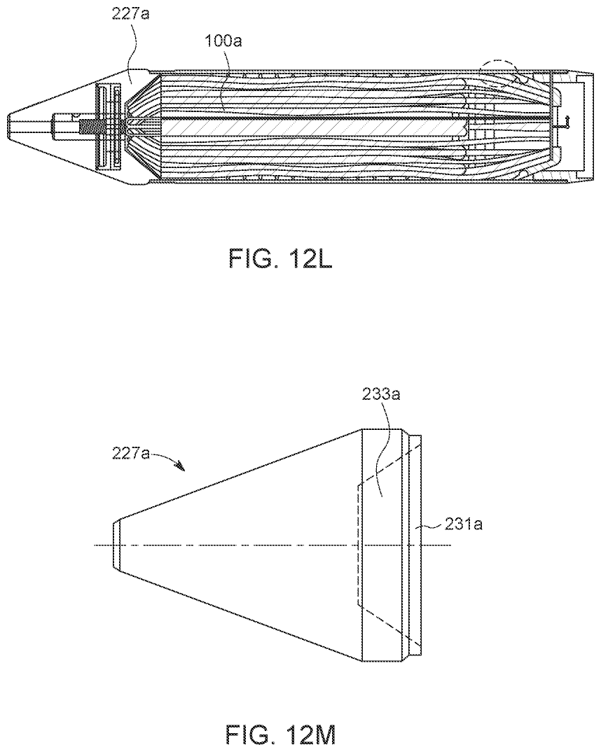

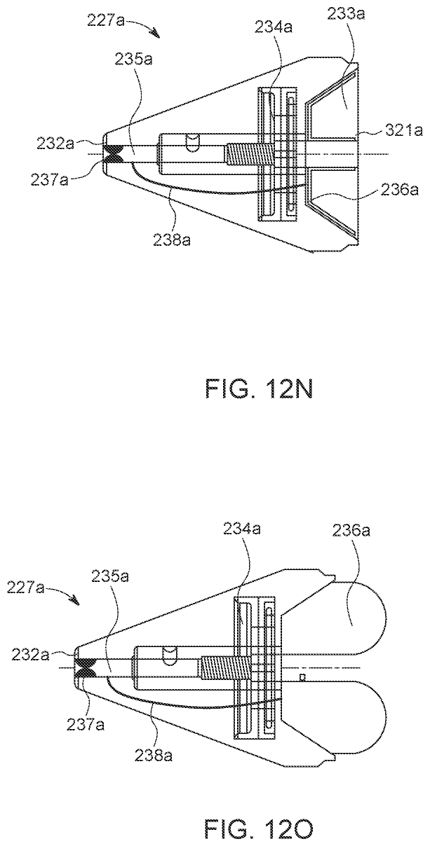

Window funnel 60 is shown more clearly in FIGS. 12A and 12B. Window funnel 60 extends from a proximal end to a distal end, wherein the proximal end is positioned relatively nearer the user and the distal end is positioned relatively farther from the user when the window funnel is in use on delivery device 200. As noted above, window funnel 60 includes a distal flange 65 at the distal end, and further includes a proximal flange 64 at the proximal end. Extending between proximal flange 64 and distal flange 65 is a body 66, which has a cylindrical exterior as shown in FIGS. 12A and 12B. Window funnel 60 defines a continuous lumen extending a full length of the window funnel, i.e., through proximal flange 64, body 66 and distal flange 65. Replacement heart valve 100 may be translated through window funnel 60 in the loading direction, which is from the distal end to the proximal end. As such, replacement heart valve 100 may be loaded into the lumen of window funnel 60 at the distal end (in some examples, from slotted funnel 190) by pulling the valve proximally via suture catheter 214 as described above.

Proximal flange 64 may abut or couple to a loading tube 222 as shown in FIG. 12A, which is a hollow transparent cylindrical element surrounding several other components of delivery device 200. Loading tube 222 may primarily function as a stabilizing device. In other words, many catheter components of delivery device 200 may be intentionally flexible, and thus those components may not easily be able to withstand proximally directed forces along their axes. For example, valve cover 223 may include slots or other features to increase flexibility during delivery. A proximally directed force on valve cover 223 may tend to cause the valve cover to flex. The loading tube 222, on the other hand, may be rigid and able to withstand proximally directed forces. Thus, when the window funnel 60 is coupled to the valve cover 223, for example via threading, the proximal flange 64 of the loading funnel abuts the distal end of the loading tube 222 so that axial forces on the window funnel 60 are transmitted to the loading tube 222. Thus, while pulling the prosthetic heart valve 100 proximally into the valve cover 223, the valve cover will tend not to bend or flex because the loading tube 222 is rigid and may provide the necessary support. Passing through loading tube 222 is the valve cover 223 coupled to and extending from an outer catheter or steering catheter 220 (shown more clearly in FIG. 12G). Disposed within and extending through a lumen of steering catheter 220 is a first inner catheter or extension catheter (not shown), which defines a lumen and is positioned over a second inner catheter or suture catheter 214, which defines a lumen and is positioned over a third inner catheter or nosecone catheter 216, which defines a lumen and is positioned over mandrel 212 as noted above. In other words, delivery device 200 may have a handle or base unit 201 at its proximal end, and mandrel 212 may extend distally from the handle or base unit through a lumen of nosecone catheter 216, which extends distally from the handle or base unit (after being applied over the distal end of the mandrel as described below) through a lumen of suture catheter 214, which extends distally from the base unit through a lumen of steering catheter 220, which also extends distally from the base unit. Each of extension catheter, suture catheter 214 and nosecone catheter 216 further extends through the lumen of window funnel 60 to assist with the loading process of valve 100. A distal end of the extension catheter includes can 221 coupled (e.g., welded) thereto. Can 221 is a hollow cylinder having a substantially closed proximal face and an open distal face adapted to receive and hold tips of atrial anchor 102 of replacement heart valve 100, as described below in greater detail. Suture catheter 214 couples to retention mechanism 180 as described above to pull valve 100 proximally through the slotted funnel 190 and then through the window funnel 60. Window funnel 60 may have an internal diameter that tapers in the loading direction. That is, the distal end of window funnel 60 may have a first internal diameter, and the internal diameter may decrease gradually as the window funnel extends proximally. The tapering diameter may further compress replacement heart valve 100 and transition the valve to the fully collapsed condition so that it may have a sufficiently small radial diameter to be loaded into can 221 and valve cover 223. Further, window funnel 60 may have a length sufficient to hold an entire length of replacement heart valve 100 in the fully collapsed condition. That is, the axial length of valve 100 increases as the valve is collapsed further, and window funnel 60 may have a length equal to or greater than the maximum axial length reached by the valve in the fully collapsed condition such that an entirety of the fully collapsed valve may fit within the window funnel at a given time in the loading process.

In addition to loading tube 222 abutting proximal flange 64 of window funnel 60, delivery device 200 includes a steering catheter 220 and valve cover 223 extending through the loading tube and generally abutting the proximal flange so that a lumen of the steering catheter is in communication with the lumen of window funnel 60. As noted above, the extension catheter includes a can 221 coupled to (e.g., laser welded, threadably, or the like) and extending from the extension catheter. During loading of valve 100 into valve cover 223, can 221 is positioned within the lumen of window funnel 60 as shown in FIG. 12A. Can 221 is visible within window funnel 60 because the window funnel has at least one, and preferably two, windows 70 carved out of body 66 of the window funnel for visibility. In the example of FIG. 12A, windows 70 are generally rectangular in shape, and window funnel 60 includes two windows opposite each other and approximately 180 degrees apart around a circumference of body 66 of the window funnel. Windows 70 allow for visibility for the user to observe and monitor the movement of valve 100 through window funnel 60 and into can 221. Windows 70 may have fully rounded edges surrounding the aperture to allow valve 100 to contact and move smoothly past the windows while the valve is translated through window funnel 60 without damaging any components of the valve. It is contemplated that the window funnel may include any number of windows of any size and shape and in any location on the window funnel to optimize visibility of the valve and delivery device components. Specifically, as valve 100 is translated proximally through window funnel 60, the valve is compressed further toward the fully collapsed condition in a manner similar to that described above with respect to the valve passing through slotted funnel 190. When valve 100 approaches a proximal portion of window funnel 60, a proximal end of the valve enters can 221.

As noted above, can 221 is sized, shaped and positioned to receive and hold tips of atrial anchor 102 of replacement heart valve 100. As valve 100 is pulled proximally through window funnel 60 by suture catheter 214, the tips of atrial anchor 102, which includes crests 122 and attachment members 118, are positioned at the proximal-most end of the valve in its collapsed condition. Thus, the atrial tips are among the first portion of valve 100 to meet and contact can 221 during loading. The movement of the atrial tips during loading may be monitored through the windows 70 of window funnel 60. Valve 100 may be translated proximally until the atrial tips are visibly seated within can 221, as shown in FIG. 12E. Once a user has visibly determined through windows 70 that the atrial tips are restrained within can 221, valve 100 may be translated further proximally by suture catheter 214 in tandem with the steering catheter so that the valve and can are transitioned from window funnel 60 into valve cover 223, as shown in FIG. 12F. Alternatively, after the atrial tips are confirmed to be loaded into can 221, window funnel 60 may be decoupled from valve cover 223 and/or loading tube 222, and the valve cover may be pushed distally relative to the valve so that the valve and can are disposed within the valve cover.

It is also contemplated that valve 100 may include markings, such as colored sutures, that are used to indicate the positioning of the valve within can 221. For example, the colored sutures on the valve may be monitored through windows 70 such that when the colored sutures are visible through the window, it signifies that valve 100 has been translated a sufficient distance for the atrial tips to be seated in can 221. Alternatively, the colored sutures may be included on or near the atrial tips so that the movement of the sutures can be clearly monitored through windows 70, and the disappearance of the colored sutures into can 221 may be construed as a sign of complete loading of the atrial tips into the can. Another indication that the atrial tips of valve 100 are loaded into can 221 may be proximal movement of the can. That is, as suture catheter 214 draws valve 100 proximally through window funnel 60, valve will be pulled proximally until the atrial tips are seated within can, and any additional proximal force applied to the valve may apply a proximal force to the can and cause proximal movement, which can thus be construed as an indication that the atrial tips are seated within the can.

When the entire length of replacement heart valve 100 is disposed within window funnel 60, and the user has confirmed that the tips of the atrial anchor 102 are positioned inside of the can 221, slotted funnel 190 may be decoupled from the window funnel and removed from the loading site, as shown in FIGS. 12B and 12C. After slotted funnel 190 is removed, window funnel 60 remains with valve 100 contained therein. It is contemplated that counterbearing 265 may be decoupled from mandrel 212 and removed from the loading site, or the counterbearing and mandrel may be decoupled from the delivery system and removed together by translating the mandrel distally out of the delivery device. Further, hypotube 250 may be removed from the delivery device by pulling the hypotube distally relative to the delivery device and decoupling proximal end 251 of the hypotube from the distal end of nosecone catheter 216 (e.g., by unthreading). Removal of hypotube 250 leaves nosecone catheter 216 extending distally from window funnel 60 as shown in FIG. 12D. Window funnel 60 may thereafter be removed and valve 100 may be disposed within valve cover 223 as described above and shown in FIG. 12G.

When the entire length of replacement heart valve 100 in the fully collapsed condition is sheathed and disposed within valve cover 223, window funnel 60 may be decoupled from loading tube 222 and/or valve cover 223, and the window funnel and loading tube may be removed from the loading site. Delivery device 200 is shown in FIG. 12G with window funnel 60 removed therefrom, the figure illustrating valve cover 223 and steering catheter 220 disposed within loading tube 222 and nosecone catheter 216 disposed within and extending distally from the valve cover. Loading tube 222 may then be decoupled and removed from valve cover 223 as shown in FIG. 12H, and a ring 225 may be applied and coupled to a distal end of the valve cover, as shown in FIG. 12I. The distal end of valve cover 223 may have external threads, and ring 225 may have internal threads to be threadably coupled to the distal end of the valve cover. As such, ring 225 covers the external threads of valve cover 223 that would otherwise remain exposed and forms a smooth external surface at the distal end of the valve cover. Ring 225 may be formed of tantalum or the like and may be used as an indicator that the valve cover 223 and/or valve 100 has been properly positioned when the valve is being deployed from the delivery device. A nosecone 227 may be placed on and coupled to (e.g., threadably or the like) the distal end of nosecone catheter 216 as shown in FIG. 12J. For example, a distal end of nosecone catheter 216 may have external threads which were threadably coupled to internal threads of hypotube 250, and after the hypotube is decoupled from the nosecone catheter, nosecone 227 may have internal threads adapted to threadably couple to the same external threads of the nosecone catheter. Nosecone 227 has a radially widened central waist portion and is generally cone-shaped between the central waist portion and the distal end, as shown in FIGS. 12J and 12K. Nosecone 227 has a bore extending longitudinally through at least a portion of the nosecone from the proximal end, the bore adapted to receive nosecone catheter 216. The bore is sized and shaped to have a diameter substantially similar to that of nosecone catheter 216 to form a secure attachment between the nosecone catheter and nosecone 227. The central waist portion of nosecone 227 has a diameter that is substantially the same as or slightly larger than a diameter of ring 225 so that the nosecone can be seated within the ring and substantially or completely fill the opening of the ring as shown in FIG. 12K. Nosecone 227 may be formed of polyurethane or other suitable material. The cone shape of nosecone 227 from the central waist portion to the distal end forms a narrow distal leading tip at the distal end of delivery device 200 that is advantageous for navigating the delivery device through the patient's vascular system to safely reach the native valve in which replacement valve 100 will be placed. Nosecone 227 may optionally be coated in barium sulfate or the like so that it is visible under x-ray.

As noted above, various replacement heart valves may be used in the described delivery procedure, and the amount of space within valve cover 223 after delivery device 200 is assembled in the delivery configuration shown in FIG. 12K may vary depending on the size of replacement heart valve 100. In some examples, replacement heart valve 100 may be sized such that the valve fits with either no remaining space or with excess space within valve cover 223 when the replacement valve is fully loaded. In other examples, replacement heart valve 100 may be sized such that valve cover 223 has insufficient space to contain the replacement valve with nosecone 227 securely coupled thereto. In such examples, alternative embodiments of delivery device 200, particularly nosecone 227, as described below may be used to comfortably fit larger sizes of replacement heart valves.

As larger replacement heart valves are created and produced having a greater axial length in the collapsed configuration, delivery device 200 may need to be modified to accommodate such lengths. While lengthening valve cover 223 may be an option, it may be preferable to maintain a minimal size of the valve cover (which is less flexible than other parts of delivery device 200) to maintain a higher degree of maneuverability. Thus, an alternative option may be to modify nosecone 227 to increase the available space within the distal end of delivery device 200. For example, as shown in FIGS. 12L and 12M, a nosecone 227a may include a cavity 233a carved out of a proximal end 231a of the nosecone. As illustrated in the fully assembled configuration in FIG. 12L, replacement heart valve 100a may extend distally beyond the proximal-most end of nosecone 227a such that a portion of the replacement heart valve is nested inside a portion of the nosecone. The general contour of nosecone 227a is shown more clearly in FIG. 12M by a dotted line. That is, the radially outer rim at proximal end 231a of nosecone 227a extends to the proximal-most end of the nosecone, whereas a radially inner portion at the proximal end of the nosecone defines a cavity 233a as shown by the dotted line. Forming cavity 233a in nosecone 227a provides additional space within valve cover 223 to fully capture a replacement heart valve 100a of greater size in the collapsed condition and still allows nosecone 227a to be fully seated within the distal opening of valve cover 223 for complete sealing to prevent air intake. In such an example, it is contemplated that an insert 234a (e.g., shown in FIGS. 12N and 12O), which has an internal threading to allow nosecone 227a to be threadably coupled to the external threading of nosecone catheter 216, may need to be positioned slightly distally within nosecone 227a relative to other embodiments using a standard nosecone 227.

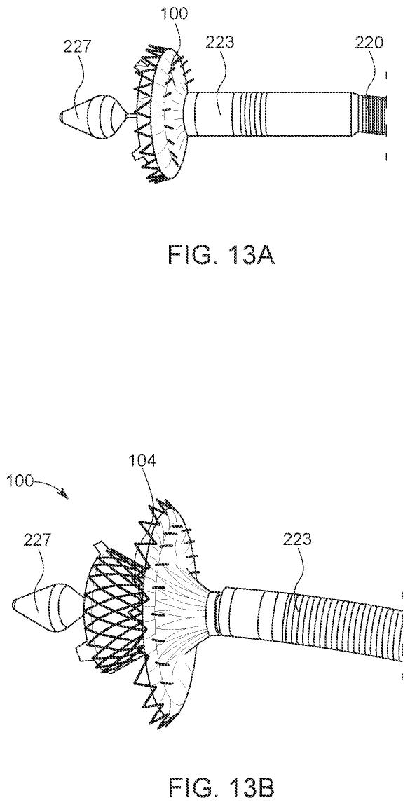

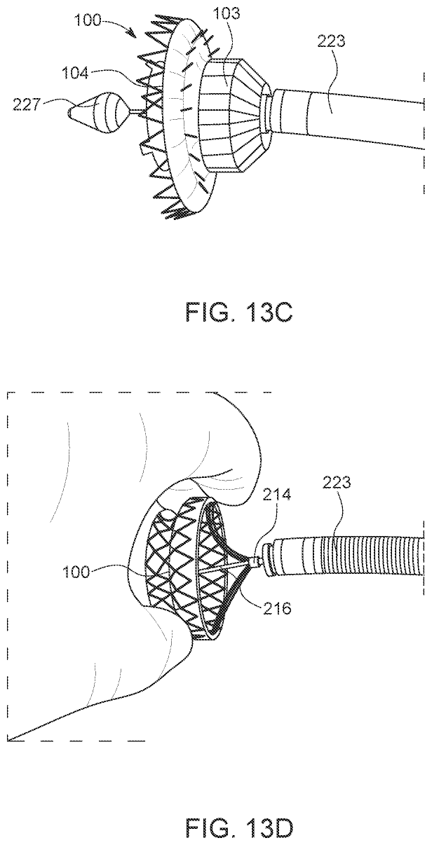

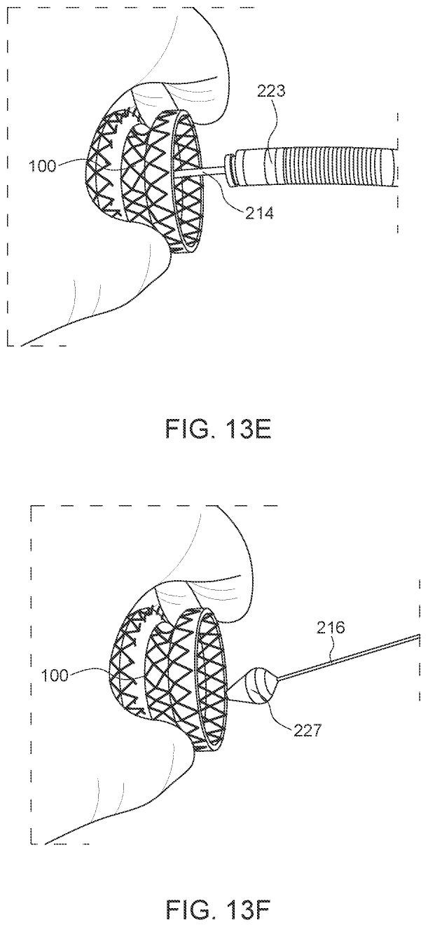

It is further contemplated that cavity 233a of nosecone 227a may interfere with replacement heart valve 100a as the valve is deployed from delivery device 200. For example, nosecone 227a is positioned distal to replacement heart valve 100a as the valve is being deployed from delivery device 200 into a native mitral annulus of a heart a shown in FIG. 13C, and the nosecone must be pulled proximally through the radial middle of the replacement valve after the valve is radially expanded. As nosecone 227a is pulled proximally through replacement heart valve 100a, cavity 233a may catch on a pointed tip of the frame of the replacement valve, such as anchor assembly 101 or strut frame 105, if the nosecone is off-centered from the replacement heart valve as the nosecone is retracted. To avoid such an interference, an inflatable member may optionally be incorporated into nosecone 227a as described below.

In some examples, such as the example illustrated in FIGS. 12N and 12O, nosecone 227a includes cavity 233a with balloon 236a disposed therein. Nosecone 227a further includes insert 234a coupled to a tube 235a which together define a lumen extending from proximal end 231a of the nosecone (e.g., cavity 233a) to distal end 232a of the nosecone. In some examples, insert 234a and tube 235a may be a single monolithic plastic piece defining the lumen through nosecone 227a. In other examples, insert 234a may be distinct from tube 235a and made of, e.g., a plastic, polymer or the like, and the tube may be a metal or polymer piece threadably coupled to the insert as illustrated in FIG. 12N. Tube 235a may have an internal valve or seal 237a at or near the tube's distal end, such as a silicon seal or the like. Seal 237a may be sufficiently flexible to allow a guidewire to pass therethrough. Nosecone 227a may further include a tube or fluid channel 238a extending from a first end to a second end, wherein the first end may be coupled to tube 235a at a position proximal to seal 237a and in fluid communication with the lumen of the tube, and the second end may be coupled to balloon 236a and in fluid communication with the interior of the balloon. As such, a fluid (such as a saline solution or the like) may be injected through the lumen of insert 234a and tube 235a toward distal end 232a of nosecone 227a. The fluid may reach seal 237a, but is substantially prevented from exiting through distal end 232a of nosecone 227a by the seal, and may thus travel into and through fluid channel 238a and enter balloon 236a. Balloon 236a is shown in FIG. 12N in a deflated condition, and sufficient fluid may be injected through nosecone 227a to expand the balloon into an inflated condition as shown in FIG. 12O. In the inflated condition, balloon 236a may contact replacement heart valve 100a as nosecone 227a is retracted through the radial middle of the expanded replacement valve during delivery to effectively prevent cavity 233a of nosecone 227a from undesirably catching on a portion of the replacement valve.

Delivery device 200 may be navigated through the body of a patient in the form shown in FIG. 12K with replacement heart valve 100 disposed therein in the fully collapsed condition to reach its delivery destination. For example, delivery device 200 may be inserted transfemorally and transseptally until the distal end of the delivery device reaches the native mitral valve annulus for delivery of replacement heart valve 100 in the native mitral valve. To begin deployment of valve 100 from delivery device 200, valve cover 223 may be translated proximally relative to nosecone 227 and the valve 100 to begin uncovering the valve. As valve cover 223 is displaced and translated proximally, replacement heart valve 100 emerges from the distal opening of the valve cover as shown in FIG. 13A. As portions of valve 100 emerge from the confines of delivery device 200, the shape-memory nature of anchor assembly 101 and strut frame 105 cause the valve to transition from the fully collapsed condition to the expanded condition. For example, as shown in FIGS. 13A and 13B, ventricular anchor 104 is deployed from delivery device 200 and at least partially expanded from the collapsed condition. As shown in FIGS. 13B and 13C, the atrial tips of valve 100 remain disposed within can 221 while the ventricular end of the valve is uncovered by valve cover 223 and deployed from delivery device 200. Can 221 continues to maintain the atrial tips in the collapsed condition while the remaining portions of the valve are transitioned to the expanded condition to assist with delivery. Valve cover 223 may continue to be drawn proximally relative to valve 100 as shown in FIG. 13C, wherein the valve continues to expand and additional portions of the valve are deployed and displaced from valve cover 223, such as central portion 103. Still further, valve cover 223 may continue to be displaced and/or valve 100 may be pushed distally until the valve is completely deployed and displaced from valve cover 223 as shown in FIG. 13D, in which the valve has returned to the completely expanded condition as it was prior to being loaded into slotted funnel 190. Suture catheter 214 may then be pushed distally relative to valve 100 so that at least a portion of the suture catheter passes through the lumen of the valve. The distal movement of suture catheter 214, which has suture loops coupled thereto, may push the ends of the suture loops coupled to the suture catheter distally relative to attachment members 118 of valve 100 (i.e., the points in which the opposite ends of each suture loop is coupled to the valve) to decouple the suture loops from their respective attachment members on the valve, as shown in FIG. 13E. After the suture loops are decoupled from the attachment members, suture catheter 214 and nosecone catheter 216 may be retracted proximally relative to valve 100, or the valve may be translated distally relative to the catheters, so that the catheters are no longer passing through the lumen of the valve, as shown in FIG. 13F.

In some examples, replacement heart valve 100 may be deployed in the left ventricle of the heart, and after at least partial or full deployment, the valve may be pulled proximally via the connection of the suture loops between suture catheter 214 and attachment members 118 to anchor the valve within the native valve annulus. Once valve 100 is seated within the native annulus and placed in the desired position, suture catheter 214 may be translated distally through the lumen of the valve to decouple the suture loops from attachment members 118, and the suture catheter may thereafter be retracted proximally to remove the suture catheter and delivery system 200 from the implantation site.

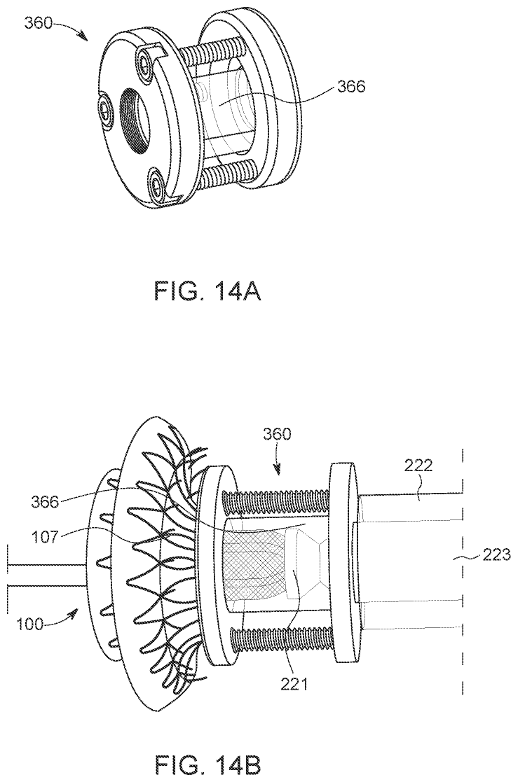

In some examples, a delivery device may optionally use a window funnel 360 having a transparent body 366 as shown in FIGS. 14A and 14B. Window funnel 360 is substantially similar to window funnel 60 described above, with the exception that window funnel 360 provides additional visibility of valve 100 to the operator as the valve is translated through the window funnel. That is, rather than viewing valve 100 through one or a plurality of windows 70, the entirety of the valve and it's passing through window funnel 360 may be viewed and monitored through transparent body 366. Such viewing may allow an operator to ensure proper collapse of valve 100 and the atrial tips into can 221. It is contemplated that body 366 may also be translucent or otherwise see-through, so long as valve 100 and its components are generally visible through the body. It is also contemplated that window funnel 360 may have a combination of a transparent body 366 and windows as described above with respect to window funnel 60. However, it should be understood that the windows or openings may be unnecessary if the body 366 is transparent. While a transparent body 366 may provide for more complete visualization, suitable materials to form the body to be transparent may be less optimal than certain opaque materials, including metals. For example, as the replacement valve 100 is being pulled through the window funnel 60 or 360, the ventricular tines 107 may scrape against the inner surface of the window funnel. Using a material such as glass for the transparent body 366 may lead to the window funnel getting scratched or otherwise damaged, and thus there may be tradeoffs between increased amount of total visualization available in the window funnel and the material used to form the window funnel.

Any and all catheters or tubes described herein may be laser cut from a metal so that they are durable and suitable for handling the forces that may be applied during delivery, particularly at the distal ends of the tubes. It is contemplated that a guidewire may be employed in the delivery process. For example, when the replacement heart valve is in the fully collapsed condition with the valve cover and is covered by the nosecone, a guidewire may extend distally from the nosecone and/or the nosecone may be adapted to pass over and travel along the guidewire to reach the delivery site of the valve. The guidewire may be formed of nitinol and have shape-memory properties so that a tapered distal end of the guidewire is pig-tailed when at rest. The guidewire may be biased to be generally straight while being passed through the veins of the patient, and as the guidewire reaches the ventricle of the patient's heart, it may return to its resting pig-tailed shape to hide its traumatic tip and prevent unintentional puncturing of the ventricle. The guidewire may be inserted transseptally and may then be passed through the middle of the native mitral valve to map out the path for the steering catheter to follow. Following a guidewire may prevent the steering catheter from hitting the chordae tendineae in the native valve, which would likely lead to entanglement.

According to a first aspect of the disclosure, a system for loading a replacement heart valve into a delivery device comprises:
a base unit positioned at a proximal end of the delivery device;
a first catheter adapted to be coupled to and extend distally from the base unit, the first catheter defining a first lumen;
a mandrel adapted to be coupled to and extend distally from the base unit, the mandrel sized to be positioned radially inward of the first catheter and extend through the first lumen;
a hypotube configured to be coupled to a distal end of the first catheter, wherein the hypotube includes an internal support member coupled thereto configured to support a replacement heart valve during a collapsing of the replacement heart valve; and
a counterbearing adapted to be coupled to a distal end of the mandrel to maintain axial positioning of the internal support member relative to the replacement heart valve while the internal support member supports a collapse of the replacement heart valve; and/or
a second catheter adapted to be coupled to and extend distally from the base unit, the second catheter defining a second lumen sized for the first catheter to extend therethrough; and/or
a third catheter adapted to be coupled to and extend distally from the base unit, the third catheter defining a third lumen sized for the second catheter to extend therethrough; and/or
a fourth catheter adapted to be coupled to and extend distally from the base unit, the fourth catheter defining a fourth lumen sized for the third catheter to extend therethrough; and/or
a valve cover positioned on a distal end of the fourth catheter, the valve cover configured to receive and maintain the replacement heart valve in a collapsed condition; and/or
a funnel adapted to be releasably coupled to a distal end of the valve cover, the funnel defining a fifth lumen extending therethrough; and/or
a distal end of the third catheter includes a valve-receiving can coupled thereto, the valve-receiving can adapted to be positioned within the funnel and to receive at least a portion of the replacement heart valve; and/or
the funnel has a body having a thickness, the body defining a first aperture extending through the thickness of the body to form a first window allowing an operator to view the fifth lumen within the funnel; and/or
the body defines a second aperture extending through the thickness of the body forming a second window, the second window positioned opposite the first window around a circumference of the body; and/or
the first and second windows are each sized and shaped to allow viewing of the replacement heart valve in the collapsed condition and prevent the replacement heart valve from transitioning from the collapsed condition to an expanded condition; and/or
the funnel has a length, and wherein the length of the funnel and a position of the first window along the body are configured to allow viewing of the valve-receiving can while the valve-receiving can is positioned within the funnel; and/or
the length of the funnel is sufficient to hold an entire length of the replacement heart valve in the collapsed condition; and/or
the first window has rounded edges adapted to contact the replacement heart valve without damaging the replacement heart valve as the replacement heart valve is translated relative to the funnel; and/or
the funnel is transparent; and/or
when the funnel is coupled to a distal end of the fourth catheter, the fifth lumen is in communication with the fourth lumen; and/or
a ring adapted to be coupled to a distal end of the valve cover; and/or
the nosecone is sized and shaped to be seated within and fill a distal opening of the valve cover; and/or
the internal support member is a balloon; and/or
a collapsible and expandable replacement heart valve.

According to a second aspect of the disclosure, an internal support member for assisting with collapsing a replacement heart valve comprises:
a first tube extending from a proximal end to a distal end;
a second tube coupled to the first tube at the distal end; and
a balloon extending from a proximal end to a distal end, wherein the proximal end of the balloon is coupled to the first tube and the distal end of the balloon is coupled to the second tube, the balloon configured to be inflated and deflated via injection and withdrawal of a fluid, wherein the proximal end of the first tube is adapted to be coupled to a catheter; and/or
the balloon is a first balloon, the internal support member further comprising a second balloon positioned radially inward of the first balloon, the second balloon configured to be inflated and deflated via injection and withdrawal of the fluid to counteract unbalanced forces applied to the internal support member; and/or
the balloon is a first balloon, the internal support member further comprising a second balloon coupled to the first tube configured to be inflated and deflated via injection of the fluid, the second balloon positioned adjacent to and proximally relative to the first balloon; and/or
the proximal end of the first tube is threaded for coupling to the catheter; and/or
the first and second tubes define a lumen therethrough sized and shaped for a mandrel to pass through the lumen; and/or
the first tube is formed of a metal; and/or
the second tube is formed of a polymer; and/or
the balloon has a length equal to or greater than a length of the replacement heart valve when the replacement heart valve is in a collapsed condition for the balloon to support the length of the replacement heart valve as the replacement heart valve is translated through a funnel to be transitioned from an expanded condition to the collapsed condition.

According to a third aspect of the disclosure, a method of loading a replacement heart valve into a delivery device comprises:
passing a mandrel through a lumen of a replacement heart valve;
coupling a first inner catheter to a retention mechanism coupled to the replacement heart valve;
positioning a second inner catheter over the mandrel such that the mandrel passes through a lumen of the second inner catheter, where the second inner catheter extends within the first inner catheter;
positioning a hypotube over the mandrel such that the mandrel passes through a lumen of the hypotube;
coupling the hypotube to the second inner catheter;
translating the first inner catheter in a proximal direction to draw the replacement heart valve through a first funnel to transition the replacement heart valve from an expanded condition to a collapsed condition over an internal support member coupled to the hypotube;
translating the first inner catheter in the proximal direction to draw the replacement heart valve from the first funnel to a second funnel;
decoupling the first funnel from the second funnel;
translating the first inner catheter in the proximal direction to draw the replacement heart valve from the second funnel to an outer catheter;
decoupling the second funnel from the outer catheter;
coupling a nosecone to the second inner catheter; and
translating the second inner catheter proximally relative to the outer catheter to seat the nosecone within an opening of the outer catheter; and/or
coupling a counterbearing to a distal end of the mandrel to abut a distal end of the hypotube to maintain positioning of the hypotube while the replacement heart valve is collapsed over the internal support member; and/or
locking the hypotube with a fluid fitting; and/or
translating the first inner catheter in the proximal direction to draw the replacement heart valve from the second funnel to the outer catheter includes monitoring a proper transition of atrial tips on the replacement heart valve into a can coupled to a distal end of a third inner catheter through a window on the second funnel; and/or
coupling a ring to a distal end of the outer catheter after decoupling the second funnel from the outer catheter.

According to a fourth aspect of the disclosure, a method of collapsing a replacement heart valve comprises:
passing a mandrel through a lumen of a funnel;
coupling an inner catheter to the replacement heart valve;
positioning a hypotube over the mandrel such that the mandrel passes through a lumen of the hypotube, the hypotube including an internal support member coupled thereto; and
drawing the inner catheter proximally to translate the replacement heart valve through the funnel to transition the replacement heart valve from an expanded condition to a collapsed condition while the internal support member is positioned radially inward of the replacement heart valve; and/or
positioning a counterbearing that is coupled to a distal end of the mandrel to abut a distal end of the hypotube to maintain positioning of the hypotube while the replacement heart valve is collapsed with the internal support member positioned radially inward of the replacement heart valve.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for loading a replacement heart valve into a delivery device, the system comprising:
a base unit positioned at a proximal end of the delivery device;
a first catheter adapted to be coupled to and extend distally from the base unit, the first catheter defining a first lumen;
a mandrel adapted to be coupled to and extend distally from the base unit, the mandrel sized to be positioned radially inward of the first catheter and extend through the first lumen;
a hypotube configured to be coupled to a distal end of the first catheter, wherein the hypotube includes an internal support member coupled thereto configured to support a replacement heart valve during a collapsing of the replacement heart valve; and
a counterbearing adapted to be coupled to a distal end of the mandrel to maintain axial positioning of the internal support member relative to the replacement heart valve while the internal support member supports a collapse of the replacement heart valve.

2. The system of claim 1 further comprising:
a second catheter adapted to be coupled to and extend distally from the base unit, the second catheter defining a second lumen sized for the first catheter to extend therethrough;
a third catheter adapted to be coupled to and extend distally from the base unit, the third catheter defining a third lumen sized for the second catheter to extend therethrough; and
a fourth catheter adapted to be coupled to and extend distally from the base unit, the fourth catheter defining a fourth lumen sized for the third catheter to extend therethrough.

3. The system of claim 2, further comprising a valve cover positioned on a distal end of the fourth catheter, the valve cover configured to receive and maintain the replacement heart valve in a collapsed condition.

4. The system of claim 3, further comprising a funnel adapted to be releasably coupled to a distal end of the valve cover, the funnel defining a fifth lumen extending therethrough.

5. The system of claim 4, wherein a distal end of the third catheter includes a valve-receiving can coupled thereto, the valve-receiving can adapted to be positioned within the funnel and to receive at least a portion of the replacement heart valve.

6. The system of claim 5, wherein the funnel has a body having a thickness, the body defining a first aperture extending through the thickness of the body to form a first window allowing an operator to view the fifth lumen within the funnel.

7. The system of claim 6, wherein the body defines a second aperture extending through the thickness of the body forming a second window, the second window positioned opposite the first window around a circumference of the body.

8. The system of claim 7, wherein the first and second windows are each sized and shaped to allow viewing of the replacement heart valve in the collapsed condition and prevent the replacement heart valve from transitioning from the collapsed condition to an expanded condition.

9. The system of claim 6, wherein the funnel has a length, and wherein the length of the funnel and a position of the first window along the body are configured to allow viewing of the valve-receiving can while the valve-receiving can is positioned within the funnel.

10. The system of claim 9 wherein the length of the funnel is sufficient to hold an entire length of the replacement heart valve in the collapsed condition.

11. A method of delivering a prosthetic heart valve, the method comprising:
advancing the prosthetic heart valve toward a native heart valve annulus while the prosthetic heart valve is maintained in a collapsed condition by a valve cover at a distal end of a catheter of a delivery device, a nosecone having a tapered distal end and a proximal end positioned adjacent to a distal end of the valve cover during the delivering, a balloon being disposed on the proximal end of the nosecone, the balloon being in a deflated condition during the advancing;
after reaching the native heart valve annulus, deploying the prosthetic heart valve into the native valve annulus by withdrawing the valve cover relative to the prosthetic heart valve and by allowing the prosthetic heart valve to expand into the native heart valve annulus, wherein while deploying the prosthetic heart valve, the proximal end of the nosecone is spaced apart from the distal end of the valve cover;
after allowing the prosthetic heart valve to expand, inflating the balloon; and
after inflating the balloon, retracting the nosecone while the balloon is inflated proximally toward the distal end of the valve cover.

12. The method of claim 11, wherein the proximal end of the nosecone defines a cavity, and the prosthetic heart valve is at least partially received within the cavity while the balloon is deflated while advancing the prosthetic heart valve.

13. The method of claim 12, wherein while advancing the prosthetic heart valve, a portion of the prosthetic heart valve extends beyond the distal end of the valve cover.

14. The method of claim 11, wherein while retracting the nosecone toward the distal end of the valve cover, the inflated balloon contacts the prosthetic heart valve.

15. The method of claim 11, wherein the nosecone defines an interior lumen, and a flexible seal is positioned within the interior lumen.

16. The method of claim 15, wherein while advancing the prosthetic heart valve to the native heart valve annulus, a guidewire extends through the interior lumen of the nosecone and across the flexible seal.

17. The method of claim 16, wherein the seal is positioned at a distal end of the interior lumen.

18. The method of claim 17, wherein inflating the balloon includes passing inflation media through a fluid channel that has a first end that leads into the interior lumen proximal to the flexible seal, and a second end that leads into an interior volume of the balloon.

19. The method of claim 11, wherein after inflating the balloon, the balloon extends beyond the proximal end of the nosecone.

20. The method of claim 11, wherein allowing the prosthetic heart valve to expand includes allowing the prosthetic heart valve to self-expand.
